SEQUENCE LISTING (17 sequences)

SEQ ID NO: 1 - protein, 437 aa, synthetic construct
  REGION 1..437: note = Hc/TriRecABTox
KNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMFNAGNKNSYIKLKKDSPVGEILGPRGSVMTTNIYLNSSLYRGEKFIIRRKSNSQSINDDIVRNEDYIYLDFFNLNQEWRVYTYKYFKKEEEKLFLAPISDSDEFYNTIQIKEYDEQGTNSCQLLFKKDEESTDEIGLIGIHRFYESGIVFEEYKDYFCISKWYLKEVKRKPYNLKLGCNWQFIPVDDGWGERPL

SEQ ID NO: 2 - DNA, 1314 bp, synthetic construct
  misc_feature 1..1314: note = Hc/TriRecABTox codon optimised sequence for expression in E. coli
  source 1..1314: mol_type = other DNA
aagaacattatcaacaccagcatcctgaacctgcgctacgagagcaaccacctgatcgacctgagccgctacgcgagcaagattaacatcggtagcaaggtgaactttgacccgattgataaaaaccagatccaactgttcaacctggaaagcagcaagatcgaagtgattctgaaaaacgcgattgtttataacagcatgtacgaaaacttcagcaccagcttttggatccgtattccgaagtattttaacagcatcagcctgaacaacgaatacaccatcattaactgcatggagaacaacagcggttggaaagtgagcctgaactacggcgaaatcatttggaccctgcaggacacccaagagatcaagcagcgtgtggttttcaagtacagccaaatgatcaacatcagcgattacatcaaccgttggattttcgttaccatcaccaacaaccgtctgaacaacagcaagatctacattaacggtcgtctgattgaccagaaaccgatcagcaacctgggcaacattcacgcgagcaacaacatcatgttcaagctggacggttgccgtgatacccaccgttatatctggattaagtacttcaacctgtttgataaagagctgaacgaaaaggagattaaagacctgtatgataaccagagcaacagcggtatcctgaaggacttttggggcgattatctgcaatacgacaaaccgtactatatgttcaacgcgggtaacaagaacagctacattaaactgaagaaagatagcccggtgggtgaaatcctgggtccgcgtggcagcgttatgaccaccaacatctatctgaacagcagcctgtaccgtggcgagaagttcatcattcgtcgtaaaagcaacagccagagcattaacgacgatatcgtgcgtaacgaagactacatttatctggatttctttaacctgaaccaagagtggcgtgtttacacctacaagtacttcaagaaagaggaagagaagctgttcctggcgccgatcagcgacagcgatgaattctacaacaccatccaaatcaaggaatacgacgagcagggtaccaacagctgccaactgctgttcaagaaagacgaagagagcaccgatgaaatcggtctgatcggcattcaccgtttctacgagagcggcatcgtgttcgaagagtacaaggattacttctgcatcagcaagtggtatctgaaagaggttaagcgtaaaccgtacaacctgaaactgggctgcaactggcaatttattccggtggatgatggctggggtgaacgtccgctgtaa

SEQ ID NO: 3 - protein, 1311 aa, synthetic construct
  REGION 1..1311: note = Full-length inactive TriRecABTox with engineered activation site
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHQLIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNAKTFLNFDKAVFKINIVPKVNYTIYDGFNLRNTNLAANFNGQNTEINNMNFTKLKNFTGLFEFYKLLCVRGIITSKTKSLIEGRDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMFNAGNKNSYIKLKKDSPVGEILGPRGSVMTTNIYLNSSLYRGEKFIIRRKSNSQSINDDIVRNEDYIYLDFFNLNQEWRVYTYKYFKKEEEKLFLAPISDSDEFYNTIQIKEYDEQGTNSCQLLFKKDEESTDEIGLIGIHRFYESGIVFEEYKDYFCISKWYLKEVKRKPYNLKLGCNWQFIPVDDGWGERPL

SEQ ID NO: 4 - DNA, 3936 bp, synthetic construct
  misc_feature 1..3936: note = Full-length inactive TriRecABTox w engineered activation site, codon opimised sequence for expression in E. coli
  source 1..3936: mol_type = other DNA
atgccgttcgtgaataagcagttcaactacaaagatccggttaatggcgtggacatcgcgtacatcaaaatcccgaatgcgggtcagatgcagccggtgaaggcgttcaaaatccacaacaaaatttgggttatcccggagcgtgacacctttaccaacccggaggaaggtgatctgaacccgccgccggaagcgaaacaagtgccggttagctactatgacagcacctatctgagcaccgacaacgagaaggataactacctgaagggcgtgaccaaactgttcgaacgtatctacagcaccgatctgggtcgtatgctgctgaccagcattgttcgtggcatcccgttttggggtggcagcaccatcgacaccgaactgaaagtgattgataccaactgcattaacgttatccagccggatggtagctaccgtagcgaggaactgaacctggtgatcattggcccgagcgcggacatcattcagtttgagtgcaagagcttcggtcacgaagttctgaacctgacccgtaacggttacggcagcacccaatatatccgtttcagcccggatttcacctttggcttcgaggaaagcctggaagtggacaccaacccgctgctgggtgcgggcaagtttgcgaccgacccggcggttaccctggcgcaccagctgatccatgcgggtcaccgtctgtacggcattgcgatcaacccgaaccgtgtgttcaaagttaacaccaacgcgtactatgagatgagcggtctggaagtgagctttgaggaactgcgtaccttcggtggccacgacgcgaagtttatcgatagcctgcaggagaacgaattccgtctgtactactacaacaagttcaaggacatcgcgagcaccctgaacaaggcgaaaagcattgtgggtaccaccgcgagcctgcaatacatgaagaacgttttcaaggagaagtacctgctgagcgaagataccagcggcaagtttagcgtggacaagctgaaattcgataagctgtataaaatgctgaccgagatctacaccgaagataacttcgtgaagttctttaaagttctgaacgcgaaaacctttctgaacttcgacaaggcggtttttaaaattaacatcgtgccgaaggttaactacaccatctatgatggtttcaacctgcgtaacaccaacctggcggcgaactttaacggccagaacaccgagattaacaacatgaactttaccaagctgaaaaacttcaccggtctgtttgaattctataaactgctgtgcgtgcgtggcatcattaccagcaagaccaaaagcctgatcgaaggtcgtgacaagggctacaacaaagcgctgaacgatctgtgcattaaagttaacaactgggacctgttctttagcccgagcgaggacaacttcaccaacgatctgaacaagggcgaggaaatcaccagcgacaccaacattgaagcggcggaggaaaacatcagcctggatctgattcagcaatattacctgacctttaacttcgacaacgagccggaaaacattagcatcgagaacctgagcagcgacatcattggtcagctggagctgatgccgaacatcgaacgtttcccgaacggcaagaaatacgaactggataaatataccatgttccactacctgcgtgcgcaagagtttgaacacggcaagagccgtattgcgctgaccaacagcgtgaacgaggcgctgctgaacccgagccgtgtttataccttctttagcagcgactacgtgaagaaagttaacaaagcgaccgaggcggcgatgttcctgggttgggtggaacagctggtttacgactttaccgatgaaaccagcgaggtgagcaccaccgacaaaattgcggatatcaccatcattatcccgtatatcggtccggcgctgaacattggcaacatgctgtacaaggacgattttgtgggtgcgctgatcttcagcggcgcggttatcctgctggagttcattccggaaattgcgatcccggtgctgggtacctttgcgctggttagctacatcgcgaacaaggtgctgaccgttcaaaccattgataacgcgctgagcaagcgtaacgagaaatgggacgaagtgtataaatacatcgttaccaactggctggcgaaggttaacacccagattgacctgatccgtaagaaaatgaaagaggcgctggaaaaccaagcggaggcgaccaaggcgattatcaactatcagtacaaccaatacaccgaggaagagaaaaacaacattaacttcaacatcgacgatctgagcagcaagctgaacgaaagcatcaacaaagcgatgattaacatcaacaagtttctgaaccagtgcagcgtgagctatctgatgaacagcatgattccgtacggtgttaagcgtctggaggacttcgatgcgagcctgaaggacgcgctgctgaaatatatctacgataaccgtggtaccctgattggccaagtggaccgtctgaaggataaagttaacaacaccctgagcaccgatatcccgttccagctgagcaaatatgtggacaaccaacgtctgctgagcacctttaccgagtacatcaagaacattatcaacaccagcattctgaacctgcgttatgaaagcaaccacctgatcgacctgagccgttacgcgagcaagattaacatcggtagcaaagttaacttcgacccgatcgataaaaaccagattcaactgtttaacctggagagcagcaagattgaagtgatcctgaaaaacgcgatcgtttacaacagcatgtatgagaactttagcaccagcttctggattcgtatcccgaaatatttcaacagcattagcctgaacaacgagtacaccattatcaactgcatggaaaacaacagcggttggaaggtgagcctgaactacggcgagattatctggaccctgcaggacacccaagaaatcaagcagcgtgtggttttcaagtacagccaaatgatcaacatcagcgattacattaaccgttggatctttgttaccattaccaacaaccgtctgaacaacagcaaaatttacatcaacggtcgtctgatcgaccagaagccgattagcaacctgggcaacatccacgcgagcaacaacattatgttcaagctggacggttgccgtgatacccaccgttatatttggatcaagtacttcaacctgttcgataaggagctgaacgagaaggaaatcaaagacctgtatgataaccagagcaacagcggtattctgaaagacttctggggcgattacctgcaatatgacaagccgtattacatgtttaacgcgggtaacaagaacagctacatcaaactgaagaaagatagcccggtgggtgaaattctgggtccgcgtggcagcgttatgaccaccaacatctatctgaacagcagcctgtaccgtggcgaaaagttcattatccgtcgtaaaagcaacagccagagcatcaacgacgatattgtgcgtaacgaggactatatctacctggatttctttaacctgaaccaagaatggcgtgtttacacctacaagtacttcaagaaagaagaggaaaagctgtttctggcgccgattagcgacagcgatgaattctataacaccattcagatcaaagagtacgacgaacagggtaccaacagctgccaactgctgtttaagaaagacgaggaaagcaccgatgagatcggtctgattggcatccaccgtttttacgaaagcggcatcgtgttcgaggaatacaaggattacttctgcatcagcaagtggtatctgaaagaggttaagcgtaaaccgtacaacctgaaactgggctgcaactggcaatttattccggtggatgatggctggggtgaacgtccgctgtaa

SEQ ID NO: 5 - protein, 1311 aa, synthetic construct
  REGION 1..1311: note = Full-length active TreRecABTox with engineered activation site
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLIEGRDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMFNAGNKNSYIKLKKDSPVGEILGPRGSVMTTNIYLNSSLYRGEKFIIRRKSNSQSINDDIVRNEDYIYLDFFNLNQEWRVYTYKYFKKEEEKLFLAPISDSDEFYNTIQIKEYDEQGTNSCQLLFKKDEESTDEIGLIGIHRFYESGIVFEEYKDYFCISKWYLKEVKRKPYNLKLGCNWQFIPVDDGWGERPL

SEQ ID NO: 6 - protein, 437 aa, synthetic construct
  REGION 1..437: note = Hc/TAB2.1, optimised loop 360
KNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMFNAGNKNSYIKLKKDSPVGEILGPRGSVMTTNIYLNSSLYRGEKFIIRRKSNSQSINDDIVRNEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPVDDGWGERPL

SEQ ID NO: 7 - DNA, 1314 bp, synthetic construct
  misc_feature 1..1314: note = Hc/TAB2.1, codon optimised for expression in E. coli
  source 1..1314: mol_type = other DNA
aagaacattatcaacaccagcatcctgaacctgcgctacgagagcaaccacctgatcgacctgagccgctacgcgagcaagattaacatcggtagcaaggtgaactttgacccgattgataaaaaccagatccaactgttcaacctggaaagcagcaagatcgaagtgattctgaaaaacgcgattgtttataacagcatgtacgaaaacttcagcaccagcttttggatccgtattccgaagtattttaacagcatcagcctgaacaacgaatacaccatcattaactgcatggagaacaacagcggttggaaagtgagcctgaactacggcgaaatcatttggaccctgcaggacacccaagagatcaagcagcgtgtggttttcaagtacagccaaatgatcaacatcagcgattacatcaaccgttggattttcgttaccatcaccaacaaccgtctgaacaacagcaagatctacattaacggtcgtctgattgaccagaaaccgatcagcaacctgggcaacattcacgcgagcaacaacatcatgttcaagctggacggttgccgtgatacccaccgttatatctggattaagtacttcaacctgtttgataaagagctgaacgaaaaggagattaaagacctgtatgataaccagagcaacagcggtatcctgaaggacttttggggcgattatctgcaatacgacaaaccgtactatatgttcaacgcgggtaacaagaacagctacattaaactgaagaaagatagcccggtgggtgaaatcctgggtccgcgtggcagcgttatgaccaccaacatctatctgaacagcagcctgtaccgtggcgagaagttcatcattcgtcgtaaaagcaacagccagagcattaacgacgatatcgtgcgtaacgaagactacatttatctggatttctttaacctgaaccaagagtggcgtgtttacacctacaagtacttcaagaaagaggaagagaagctgttcctggcgccgatcagcgacagcgatgaattctacaacaccatccaaatcaaggaatacgacgagcagccgacctatagctgccaactgctgttcaagaaagacgaagagagcaccgatgaaatcggtctgatcggcattcaccgtttctacgagagcggcatcgtgttcgaagagtacaaggattacttctgcatcagcaagtggtatctgaaagaggttaagcgtaaaccgtacaacctgaaactgggctgcaactggcaatttattccggtggatgatggctggggtgaacgtccgctgtaa

SEQ ID NO: 8 - protein, 432 aa, synthetic construct
  REGION 1..432: note = Hc/TAB2.1.1
TSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMFNAGNKNSYIKLKKDSPVGEILGPRGSVMTTNIYLNSSLYRGEKFIIRRKSNSQSINDDIVRNEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPVDDGWGERPL

SEQ ID NO: 9 - DNA, 1299 bp, synthetic construct
  misc_feature 1..1299: note = Hc/TAB2.1.1, codon optimised for expression in E. coli
  source 1..1299: mol_type = other DNA
accagcatcctgaacctgcgctacgagagcaaccacctgatcgacctgagccgctacgcgagcaagattaacatcggtagcaaggtgaactttgacccgattgataaaaaccagatccaactgttcaacctggaaagcagcaagatcgaagtgattctgaaaaacgcgattgtttataacagcatgtacgaaaacttcagcaccagcttttggatccgtattccgaagtattttaacagcatcagcctgaacaacgaatacaccatcattaactgcatggagaacaacagcggttggaaagtgagcctgaactacggcgaaatcatttggaccctgcaggacacccaagagatcaagcagcgtgtggttttcaagtacagccaaatgatcaacatcagcgattacatcaaccgttggattttcgttaccatcaccaacaaccgtctgaacaacagcaagatctacattaacggtcgtctgattgaccagaaaccgatcagcaacctgggcaacattcacgcgagcaacaacatcatgttcaagctggacggttgccgtgatacccaccgttatatctggattaagtacttcaacctgtttgataaagagctgaacgaaaaggagattaaagacctgtatgataaccagagcaacagcggtatcctgaaggacttttggggcgattatctgcaatacgacaaaccgtactatatgttcaacgcgggtaacaagaacagctacattaaactgaagaaagatagcccggtgggtgaaatcctgggtccgcgtggcagcgttatgaccaccaacatctatctgaacagcagcctgtaccgtggcgagaagttcatcattcgtcgtaaaagcaacagccagagcattaacgacgatatcgtgcgtaacgaagactacatttatctggatttctttaacctgaaccaagagtggcgtgtttacacctacaagtacttcaagaaagaggaagagaagctgttcctggcgccgatcagcgacagcgatgaattctacaacaccatccaaatcaaggaatacgacgagcagccgacctatagctgccaactgctgttcaagaaagacgaagagagcaccgatgaaatcggtctgatcggcattcaccgtttctacgagagcggcatcgtgttcgaagagtacaaggattacttctgcatcagcaagtggtatctgaaagaggttaagcgtaaaccgtacaacctgaaactgggctgcaactggcaatttattccggtggatgatggctggggtgaacgtccgctgtaa

SEQ ID NO: 10 - protein, 424 aa, synthetic construct
  REGION 1..424: note = Hc/TAB2.1.3, increased solubility
TSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMFNAGNKNSYIKLKKDSPVGEILGPRGSVMTTNIYLNSSLYRGEKFIIRRKSNSQSINDDIVRNEDYIYLDFFNLNQEWRVYTYKYFKKEEEKLFLAPISDSDEFYNTIQIKEYDEQPTYSCQLLFKKDEESTDEIGLIGIHRFNNKDYFCISKWYLKEVKRKPYNLKLGCNWQFIPVDDGWGERPL

SEQ ID NO: 11 - DNA, 1275 bp, synthetic construct
  misc_feature 1..1275: note = Hc/TAB2.1.3, codon optimised for expression in E. coli
  source 1..1275: mol_type = other DNA
accagcatcctgaacctgcgctacgagagcaaccacctgatcgacctgagccgctacgcgagcaagattaacatcggtagcaaggtgaactttgacccgattgataaaaaccagatccaactgttcaacctggaaagcagcaagatcgaagtgattctgaaaaacgcgattgtttataacagcatgtacgaaaacttcagcaccagcttttggatccgtattccgaagtattttaacagcatcagcctgaacaacgaatacaccatcattaactgcatggagaacaacagcggttggaaagtgagcctgaactacggcgaaatcatttggaccctgcaggacacccaagagatcaagcagcgtgtggttttcaagtacagccaaatgatcaacatcagcgattacatcaaccgttggattttcgttaccatcaccaacaaccgtctgaacaacagcaagatctacattaacggtcgtctgattgaccagaaaccgatcagcaacctgggcaacattcacgcgagcaacaacatcatgttcaagctggacggttgccgtgatacccaccgttatatctggattaagtacttcaacctgtttgataaagagctgaacgaaaaggagattaaagacctgtatgataaccagagcaacagcggtatcctgaaggacttttggggcgattatctgcaatacgacaaaccgtactatatgttcaacgcgggtaacaagaacagctacattaaactgaagaaagatagcccggtgggtgaaatcctgggtccgcgtggcagcgttatgaccaccaacatctatctgaacagcagcctgtaccgtggcgagaagttcatcattcgtcgtaaaagcaacagccagagcattaacgacgatatcgtgcgtaacgaagactacatttatctggatttctttaacctgaaccaagagtggcgtgtttacacctacaagtacttcaagaaagaggaagagaagctgttcctggcgccgatcagcgacagcgatgaattctacaacaccatccaaatcaaggaatacgacgagcagccgacctatagctgccaactgctgttcaagaaagacgaagagagcaccgatgaaatcggtctgatcggcattcaccgtttcaacaacaaggattacttctgcatcagcaagtggtatctgaaagaggttaagcgtaaaccgtacaacctgaaactgggctgcaactggcaatttattccggtggatgatggctggggtgaacgtccgctgtaa

SEQ ID NO: 12 - protein, 1343 aa, synthetic construct
  REGION 1..1343: note = BoNT/TAB2.1.3, full-length active protein
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHALIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNAKTFLNFDKAVFKINIVPKVNYTIYDGFNLRNTNLAANFNGQNTEINNMNFTKLKNFTGLFEFYKLLCVRGIITSKAGAGKSLVPRGSAGAGALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMFNAGNKNSYIKLKKDSPVGEILGPRGSVMTTNIYLNSSLYRGEKFIIRRKSNSQSINDDIVRNEDYIYLDFFNLNQEWRVYTYKYFKKEEEKLFLAPISDSDEFYNTIQIKEYDEQGTNSCQLLFKKDEESTDEIGLIGIHRFYESGIVFEEYKDYFCISKWYLKEVKRKPYNLKLGCNWQFIPVDDGWGERPLVPRGSANSSSVDKLWSHPQFEKLEHHHHHH

SEQ ID NO: 13 - DNA, 4032 bp, synthetic construct
  misc_feature 1..4032: note = BoNT/TAB2.1.3, sequence included in pEt29(a) vector for expression in E. coli
  source 1..4032: mol_type = other DNA
atgccatttgtgaacaagcagtttaactataaggacccggtgaacggtgtggatatcgcgtatatcaaaatcccgaatgcgggccagatgcaaccagtcaaggcgttcaagattcataacaagatttgggttattccggaacgtgataccttcaccaatccggaagaaggcgacttaaacccgccgccagaagccaaacaagtgccggtgagctactatgatagcacgtatcttagcaccgataatgaaaaagacaattacctgaagggcgtgaccaagttgttcgagcgcatctacagtaccgacttaggccgcatgttgttgacgagcatcgttcgcggtatcccgttctggggcggctcgaccattgataccgagttgaaagtcattgacacgaactgtatcaatgttatccaaccggacggcagttatcgcagcgaggagttaaatttggtcatcatcggtccaagcgcagatattattcagttcgaatgcaagagcttcggccatgaggtcttgaatttgacgcgcaacggttacggcagcacccaatacatccgctttagcccggatttcacctttggcttcgaggagagcttggaggtggacaccaacccgctgttaggtgccggcaaattcgcaaccgacccggcagtgacgttggcgcacgcgttgattcatgcgggtcaccgcttatacggtatcgcgatcaatccgaatcgcgtctttaaagtcaataccaacgcgtactacgaaatgagcggcttagaggttagctttgaagaattacgcaccttcggtggccacgacgccaagttcatcgacagcctgcaggaaaatgagttccgcttgtactattacaataaattcaaggacatcgcgagcaccttaaataaagcaaagagcattgtgggcaccaccgcaagcttgcagtacatgaagaacgtatttaaggaaaaatatttgttgtcggaggataccagcgggaaattcagcgtcgataagctgaaattcgacaaattgtataaaatgctgaccgagatttacaccgaggataacttcgtcaagttttttaaggtgttaaatgcgaagacctttttaaactttgataaagcggtgtttaaaattaatatcgtgccgaaggtgaattacaccatctacgatggtttcaatttacgcaacacgaatctggcggcgaattttaatggccaaaacaccgaaattaacaacatgaactttacgaagttaaagaatttcacgggcttattcgaattctacaagttattatgcgtgcgcggcatcattaccagcaaggcaggtgcgggcaagtccttggttccgcgtggcagcgccggcgccggcgcgctcaatgatctgtgtattaaagtcaataactgggacctgttcttcagcccgagcgaggataactttaccaacgacttaaacaaaggcgaggagatcacgagcgatacgaacatcgaggcggcggaggaaaatattagcctggacctcattcagcagtactatctgacgttcaattttgacaatgagccggagaacatcagcattgaaaatctcagcagcgacatcatcggtcagttggaactgatgccgaacattgaacgctttccgaacggcaaaaaatatgaactggacaagtataccatgttccattacttacgcgcacaggaatttgagcacggcaagagccgcattgcgctgaccaatagcgttaacgaggccttgttaaatccgagccgtgtctacacgttcttcagcagcgattatgtcaaaaaagtgaacaaggcgaccgaagccgcgatgtttttgggctgggtcgagcaattggtttacgattttaccgacgaaaccagcgaggtgagcacgaccgacaaaattgcagatatcaccatcatcattccgtacatcggtccggcgctcaatatcggcaatatgttatacaaggacgactttgtgggcgcgctgatctttagcggcgcggttatcttattagaattcatcccggagatcgcaatcccggtcttgggcacctttgcgttggtgagctatatcgcgaataaagtgctcacggtccaaaccatcgataacgcgctcagcaagcgtaatgagaaatgggacgaggtttataagtatatcgtgaccaactggttagcaaaagtcaatacgcagatcgatctcatccgcaaaaaaatgaaagaagccttggaaaatcaagcggaggcaaccaaagccatcattaattaccagtataaccaatataccgaagaagaaaaaaacaatatcaacttcaatatcgatgatttgagcagcaaactgaacgagagcattaacaaagcgatgattaacatcaacaagttcttgaatcaatgcagcgtgagctatctcatgaacagcatgatcccgtatggcgtcaaacgcttggaagattttgacgccagcctgaaagatgcgctcctcaagtatatttatgacaaccgcggcaccctcattggccaggtggaccgcttgaaggataaagtgaacaatacgctcagcacggatatcccgttccagctgagcaagtacgtcgacaaccagcgcttactgagcacctttaccgagtatatcaagaacatcattaataccagcatcctcaacttgcgctatgagagcaatcacctgatcgacctcagccgctacgccagcaagatcaacatcggcagcaaggtcaatttcgacccgatcgataagaatcagatccaattgtttaacctggaaagcagcaagatcgaggttatcttgaagaacgcgattgtgtacaacagcatgtatgagaactttagcaccagcttctggattcgtatcccgaaatatttcaacagcattagcctgaacaacgagtacaccattatcaactgcatggaaaacaacagcggttggaaggtgagcctgaactacggcgagattatctggaccctgcaggacacccaagaaatcaagcagcgtgtggttttcaagtacagccaaatgatcaacatcagcgattacattaaccgttggatctttgttaccattaccaacaaccgtctgaacaacagcaaaatttacatcaacggtcgtctgatcgaccagaagccgattagcaacctgggcaacatccacgcgagcaacaacattatgttcaagctggacggttgccgtgatacccaccgttatatttggatcaagtacttcaacctgttcgataaggagctgaacgagaaggaaatcaaagacctgtatgataaccagagcaacagcggtattctgaaagacttctggggcgattacctgcaatatgacaagccgtattacatgtttaacgcgggtaacaagaacagctacatcaaactgaagaaagatagcccggtgggtgaaattctgggtccgcgtggcagcgttatgaccaccaacatctatctgaacagcagcctgtaccgtggcgaaaagttcattatccgtcgtaaaagcaacagccagagcatcaacgacgatattgtgcgtaacgaggactatatctacctggatttctttaacctgaaccaagaatggcgtgtttacacctacaagtacttcaagaaagaagaggaaaagctgtttctggcgccgattagcgacagcgatgaattctataacaccattcagatcaaagagtacgacgaacagggtaccaacagctgccaactgctgtttaagaaagacgaggaaagcaccgatgagatcggtctgattggcatccaccgtttttacgaaagcggcatcgtgttcgaggaatacaaggattacttctgcatcagcaagtggtatctgaaagaggttaagcgtaaaccgtacaacctgaaactgggctgcaactggcaatttattccggtggatgatggctggggtgaacgtccactagtgccacgcggttccgcgaattcgagctccgtcgacaagctttggagccacccgcagttcgaaaaactcgagcaccaccaccaccaccactga

SEQ ID NO: 14 - protein, 21 aa, synthetic construct
  REGION 1..21: note = hSytI peptide
GEGKEDAFSKLKEKFMNELHK

SEQ ID NO: 15 - protein, 5 aa, synthetic construct
  REGION 1..5: note = Synthetic
  VARIANT 3: note = Xaa can be any naturally occurring amino acid
LPXTG

SEQ ID NO: 16 - protein, 426 aa, Clostridium botulinum
  SITE 1..426: note = Hc/A
KNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 17 - protein, 434 aa, Clostridium botulinum
  SITE 1..434: note = Hc/B
SEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

(19) United States
(12) Patent Application Publication
(10) Pub. No.: US 2025/0034540 A1
(43) Pub. Date: Jan. 30, 2025

(54) BOTULINUM NEUROTOXIN BIOHYBRID

(71) Applicant: TOXOTECH AB, Jãrfâlla (SE)

(72) Inventors: Pål Stenmark, Jãrfâlla (SE); Geoffrey Masuyer, Åkersberga (SE)

(21) Appl. No.: 18/615,341

(22) Filed: Mar. 25, 2024

Related U.S. Application Data
(63) Continuation of application No. 16/975,308, filed on Aug. 24, 2020, now abandoned.

(30) Foreign Application Priority Data
Feb. 26, 2018 (SE) 1850213-8

(51) Int. Cl. C12N 9/52; A61K 8/66; A61K 38/48; A61Q 19/08; C07K 14/33; C12N 15/62

(52) U.S. Cl. CPC C12N 9/52; A61K 8/66; A61K 38/4893; A61Q 19/08; C07K 14/33; C12N 15/62; C12Y 304/24069; A61K 2800/91; C07K 2319/55

(57) ABSTRACT

The present invention relates to a novel botulinum neurotoxin (BoNT) Heavy Chain Binding domain (He/TAB) adapted to synergistically bind to a synaptotagmin (Syt) receptor, a synaptic associated vesicle 2 (SV2) receptor and a ganglioside (Gang) receptor, as well as polypeptides comprising said novel He/TAB, vectors encoding said polypeptides, and uses thereof.

### CROSS-REFERENCE TO RELATED APPLICATIONS

This application is a Continuation Application of U.S. patent application Ser. No.: 16/975,308, filed Aug. 24, 2020, which is a U.S. National Phase of International Application No. PCT/EP2019/054310, filed Feb. 21, 2019, which claims priority to Swedish Application No. 1850213-8 filed Feb. 26, 2018, the contents of which are incorporated herein by reference.

### INCORPORATION BY REFERENCE OF MATERIAL SUBMITTED ELECTRONICALLY

The application contains a Sequence Listing which has been submitted electronically in .XML format and is hereby incorporated by reference in its entirety. Said .XML copy, created on Apr. 18, 2024, is named “136704-00103.xml” and is 42,685 bytes in size. The sequence listing contained in this .XML file is part of the specification and is hereby incorporated by reference herein in its entirety.

### TECHNICAL FIELD

The present invention relates to Botulinum neurotoxin polypeptides and in particular to a chimeric Botulinum neurotoxin Heavy Chain.

### BACKGROUND ART

The botulinum neurotoxins (BoNTs) are the most potent protein toxins known to man, and the causative agent of the rare paralytic disease, botulism. This family of bacterial toxins consists of eight serotypes, BoNT/A-G, and the recently described BoNT/X (Montal, 2010; Zhang et al., 2017). They all share a common architecture and are expressed as a protein of 150 kDa that is post-translationally cleaved into a di-chain molecule composed of a light chain (LC, 50 kDa), linked by a single disulphide bridge to the heavy chain (HC, 100 kDa). The HC holds two of the functional domain, with the N-terminal translocation domain (H_{N}) and the C-terminal binding domain (H_{C}), while LC is responsible for intracellular catalytic activity. BoNTs first recognise the cholinergic nerve terminals via specific cell surface receptors, and are then endocytosed within a vesicle. The acidic endosomal environment causes a conformational change that allows translocation of LC within the cytosol, also named toxin translocation. The freed catalytic domain, a zinc-protease, can then specifically target one of three neuronal SNAREs (soluble N-ethylmaleimide sensitive factor attachment protein receptors): BoNT/A, /C and /E cleave SNAP-25; BoNT/B, /D, /F, /G and /X target VAMP (synaptobrevin); syntaxin is cleaved by BoNT/C (Schiavo et al., 2000; Zhang et al., 2017). These three proteins form a complex that mediates the fusion of synaptic vesicle to the plasma membrane (Sudhof and Rothman, 2009). Proteolysis of any of the SNAREs inhibits exocytosis and thus the release of neurotransmitters, effectively causing the flaccid paralysis symptomatic of botulism (Rossetto et al., 2014). The sequence of the three functional domains has previously been described (Lacy D B, et al. 1999.). The catalytic domain is composed of the amino acids 1-437, the translocation domain of amino acids 448-872, and the binding domain of amino acids 873-1295, referring to the BoNT/A sequence in Lacy D B, et al. As all BoNT serotypes and their subtypes are homologous to a large degree, the position of the corresponding domains in any other serotype or subtype will be very similar.

The high potency of these toxins makes them an extremely useful therapeutic agent in the treatment of an increasing range of neuromuscular disorders such as strabismus, cervical dystonia and blepharospasm, as well as other conditions involving the release of acetylcholine such as hyperhydrosis (Chen, 2012). BoNT/A and /B are the only serotypes approved and commercially available as therapeutics. BoNT/A is generally considered to have a higher efficacy in humans and is therefore the serotype of choice in most cases (Bentivoglio et al., 2015). However, treatment with BoNT usually requires repeated injections, as the therapeutic effects of the toxins are only transient. This reportedly led to the emergence of resistance in a small subset of patients developing an immune response to BoNT/A (Lange et al., 2009; Naumann et al., 2013). While BoNT/B represents an alternative, its lower efficacy means that higher doses are required and thus represents a greater risk of immunogenicity (Dressler and Bigalke, 2005). In addition, BoNT/B is also associated with several adverse outcomes such as painful injections, shorter duration of action and more frequent side effects (Bentivoglio et al., 2015). The major adverse effects are also often associated with treating muscle spasms, but not cosmetic applications. This is because the adverse effects are largely due to diffusion of toxins to other regions of the body and the possibility of toxin diffusion is directly related to injected doses. The adverse effects ranges from transient non-serious events such as ptosis and diplopia to life-threatening events, even death.

The binding of BoNT/A and /B to neurons has been characterised in details, and is based on a dual-receptor mechanism, involving a synaptic vesicle protein and a ganglioside anchored on the neuronal membrane. The protein receptor for BoNT/A was identified as SV2 (Dong et al., 2006, Mahrhold et al., 2006). More precisely, BoNT/A can bind to several human SV2 isoforms A, B and C, although the toxin only recognise the N-glycosylated forms of SV2A and SV2B (Yao et al., 2016). The protein receptor for BoNT/B is synaptotagmin (Syt) (Nishiki et al., 1994, 1996; Dong et al., 2003), with a preference for Syt1 over Syt2 in humans (Strotmeier et al., 2012). Ganglioside recognition is the first step of the intoxication process for all BoNTs (Binz and Rummel, 2009), and is mediated by a shared binding mechanism centred on the conserved motif H . . . SxWY . . . G in their sequence. BoNT/A prefers binding to the terminal N-acetylgalactosamine—galactose moiety of GT1b and GD1a (Takamizawa et al. 1986; Schengrund et al. 1991), while data on BoNT/B suggest a preference for the disialyl motif of GD1b and GT1b. The different serotypes vary in their carbohydrate specificity and affinity (Rummel, 2013).

The modular arrangement and distinctive properties of the various BoNT serotypes have made the toxins a target of choice for protein engineering. In particular, several studies have showed that it was possible to swap whole domains between serotypes (Masuyer et al., 2014) and thus obtaining new toxins with unique pharmaceutical potential. For example several molecules consisting of the binding domain of BoNT/B associated with the translocation and catalytic domains of BoNT/A have been produced (Rummel et al., 2011; Wang et al., 2012; Kutschenko et al., 2017).

These so-called chimeric toxins presented attractive pharmacological properties in terms of efficacy and duration of activity, which were associated with the high affinity of BoNT/B for synaptotagmin and the higher expression of this receptor on neurons compared to SV2 (Takamori et al., 2006; Wilhelm et al., 2014).

### SUMMARY OF THE INVENTION

Because both the generation of neutralizing antibodies and toxin diffusion are directly related to injected doses, lowering toxin doses, while maintaining the same levels of toxin activity, is highly desired, which means the efficacy of individual toxin molecules has to be enhanced. It is therefore an object of the present invention to provide BoNT polypeptides with improved duration and potency, and with less risk of spreading from the site of injection. The inventors have identified a key problem with the previous attempts mentioned above in engineering chimeric BoNT polypeptides. None of the previous attempts took the structural aspect of the polypeptide into account.

Using a structure-based approach and the current knowledge on the receptor binding mechanisms of BoNT/A and /B, the inventors have engineered a new molecule, TriRecABTox (BoNT/TAB) comprising a specifically engineered H_{C} domain (H_{C}/TAB) that is able to recognise a SV2C receptor, a synaptotagmin receptor and a ganglioside receptor. The inventors show that BoNT/TAB can be recombinantly expressed and purified. Using X-ray crystallography, the inventors further demonstrate that BoNT/TAB can bind to its three receptors simultaneously. Thus, BoNT/TAB should recognise neuronal cells with enhanced affinity and has the potential to be a high-efficacy alternative to BoNT/A treatment.

The object above is thus attained by in a first aspect providing a botulinum neurotoxin (BoNT) Heavy Chain Binding domain (H_{C}/TAB), wherein the H_{C}/TAB comprises a) a synaptotagmin (Syt) receptor binding site, and b) a synaptic associated vesicle 2 (SV2) receptor binding site, and c) a ganglioside (Gang) receptor binding site, and wherein said H_{C}/TAB is adapted to synergistically bind to a synaptotagmin (Syt) receptor, a synaptic associated vesicle 2 (SV2) receptor and a ganglioside (Gang) receptor.

The H_{C}/TAB has a N-terminal end (H_{CN}) and a C-terminal end (H_{CC}). According to one embodiment the H_{CC} domain is composed interchangeably of sequences from BoNT serotype A (BoNT/A) and BoNT serotype B (BoNT/B).

According to a further embodiment said H_{CC} end is composed according to a sequence A₁B₁A₂B₂A₃, where A indicates a sequence from BoNT/A, and B indicates a sequence from BoNT/B.

According to yet a further embodiment the sequences of B₁, A₂ and B₂ comprise mutations and/or deletions to create stable intramolecular interfaces for the entire H_{C}/TAB.

According to yet a further embodiment the sequences forming the Gang receptor binding site originate from any Gang-receptor binding BoNT serotype and their subtypes.

According to yet a further embodiment the sequences forming the Gang receptor binding site originate from BoNT/B.

According to yet a further embodiment the sequences forming the Gang receptor binding site are located in B₂.

According to yet a further embodiment the sequences forming the Syt receptor binding site originate from any Syt receptor-binding BoNT serotype and their subtypes.

According to yet a further embodiment the sequences forming the Syt receptor binding site originate from BoNT B, DC or G.

According to yet a further embodiment the sequences forming the Syt receptor binding site are located in B₁ and B₂.

According to yet a further embodiment the HCN sequence originates from any SV2-receptor binding BoNT serotype and their subtypes

According to yet a further embodiment the H_{CN} sequence originates from BoNT/A.

According to yet a further embodiment the sequences forming the SV2 receptor binding site are located in H_{CN} and in A₁ and A₃ in the H_{CC}.

According to yet a further embodiment the H_{C}/TAB has an amino acid sequence which is at least 60%, 65%, 70%, 75%, 80%, 85%, 90%, or 95% identical to the sequence of any of the SEQ. ID.No's 1, 3, 5, 6, 8, 10 or 12.

According to a second aspect, there is provided a polypeptide comprising the H_{C}/TAB according to the first aspect and any embodiment of the first aspect, coupled to any other protein, polypeptide, amino acid sequence or fluorescent probe, directly or via a linker.

According to an embodiment of the second aspect, said polypeptide is a BoNT polypeptide (BoNT/TAB), characterized in that said BoNT/TAB in addition to the H_{C}/TAB comprises a Heavy Chain Translocation domain (H_{N}), a Light chain (LC) and an protease site positioned between the LC and H_{N} in the polypeptide sequence, wherein the H_{N} and the LC, respectively and independently of each other, originate from any of the BoNT serotypes A, B, C, D, DC, E, En, F, G or X and their subtypes, as well as BoNT-like polypeptides.

According to a further embodiment, the polypeptide may comprise any other protein, polypeptide, amino acid sequence or fluorescent probe, linked thereto directly or via a linker.

According to yet a further embodiment the protease site is an exoprotease site. According to yet a further embodiment the exprotease site is a Factor Xa site.

According to yet a further embodiment the polypeptide according the second aspect has an amino acid sequence which is at least 60%, 65%, 70%, 75%, 80%, 85%, 90%, or 95% identical to the sequence of any of the SEQ. ID. No's 1, 3, 5, 6, 8, 10 or 12.

According to a third aspect is provided a vector comprising a nucleic acid sequence encoding a H_{C}/TAB according to the first aspect and any embodiment of the first aspect, or the polypeptide according to the second aspect and any embodiment of the second aspect.

According to a fourth aspect is provided for the use of the H_{C}/TAB according to the first aspect and any embodiment of the first aspect, or the polypeptide according to the second aspect and any embodiment of the second aspect, in a therapeutic method or in a cosmetic method.

According to one embodiment of the fourth aspect, the therapeutic method or cosmetic method is a treatment to dampen and/or inactivate muscles.

According to a further embodiment of the fourth aspect, the therapeutic method is treatment and/or prevention of a disorder chosen from the group comprising neuromuscular disorders, conditions involving the release of acetylcholine, and spastic muscle disorders.

According to yet a further embodiment the disorder is chosen from the group comprising of spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, ties, tremors, bruxism, anal fissure, achalasia, dysphagia and other muscle tone disorders and other disorders characterized by involuntary movements of muscle groups, lacrimation, hyperhydrosis, excessive salivation, excessive gastrointestinal secretions, secretary disorders, pain from muscle spasms, headache pain, sports injuries, and depression.

According to yet a further embodiment the H_{C}/TAB according to the first aspect and any embodiment of the first aspect, or the polypeptide according to the second aspect and any embodiment of the second aspect, may be used in a pharmacological test, to investigate the role of said protein, polypeptide, amino acid sequence or fluorescent probe in a synaptic process.

According to yet a further embodiment the H_{C}/TAB according to the first aspect and any embodiment of the first aspect, or the polypeptide according to the second aspect and any embodiment of the second aspect, may be used as a vehicle for effectively transporting any protein, polypeptide amino acid sequence or fluorescent probe coupled thereto to a neuronal surface.

According to yet a further embodiment the H_{C}/TAB according to the first aspect and any embodiment of the first aspect, or the polypeptide according to the second aspect and any embodiment of the second aspect, may be used as a vehicle for effectively transporting any protein, polypeptide amino acid sequence or fluorescent probe into a neuronal cytosol using a toxin translocation system.

According to a fifth aspect is provided a pharmaceutical or cosmetic composition comprising the H_{C}/TAB according to the first aspect and any embodiment of the first aspect, or the polypeptide according to the second aspect and any embodiment of the second aspect.

According to one embodiment of the fifth aspect, the composition may further comprise pharmaceutically and/or cosmetically acceptable excipients, carriers or other additives.

According to a sixth aspect is provided a kit of parts comprising the composition of the fifth aspect and directions for therapeutic administration of the composition.

According to a seventh aspect is provided a method of treating a condition associated with unwanted neuronal activity, the method comprising administering a therapeutically effective amount of the H_{C}/TAB according to the first aspect and any embodiment of the first aspect, or the polypeptide according to the second aspect and any embodiment of the second aspect, or composition of the fifth aspect, to a subject to thereby treat the condition, wherein the condition is chosen from the group comprising of spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, ties, tremors, bruxism, anal fissure, achalasia, dysphagia and other muscle tone disorders and other disorders characterized by involuntary movements of muscle groups, lacrimation, hyperhydrosis, excessive salivation, excessive gastrointestinal secretions, secretary disorders, pain from muscle spasms, headache pain, sports injuries, and depression, and dermatological or aesthetic/cosmetic conditions.

### BRIEF DESCRIPTION OF THE DRAWINGS

FIG. 1: Structural information on receptor binding by BoNT/A and /B. (FIG. 1a) Superposition of the crystal structures of the binding domain of BoNT/A in complex with GT1b (PDB 2VU) and with human glycosylated SV2C (PDB 5JLV). (FIG. 1b) Crystal structure of the binding domain of BoNT/B in complex with GD1a and rat synaptotagmin2 (PDB 4KBB). Proteins represented in ribbon mode and carbohydrates as sticks. (FIG. 1c) Sequence alignment of H_{C}/A (Uniprot P10845) (SEQ ID NO: 16) and /B (Uniprot P10844) (SEQ ID NO: 17) where secondary structural elements are also provided (figure prepared with ESPript3.0; Robert and Gouet, 2014). Regions directly involved in receptor binding are highlighted for each domain with line above H_{C}/A sequence for SV2 receptor, and below H_{C}/B sequence for Syt receptor; ganglioside receptor binding site is underlined with a striped grey line.

FIG. 2: Sequence alignment of H_{C}/TAB (SEQ ID NO: 1) with receptor binding by H_{C}/A (SEQ ID NO: 16) and /B (SEQ ID NO: 17).

Protein sequences were aligned with ClustalO (Sievers et al., 2011). The segments of H_{C}/A and H_{C}/B used in the design of H_{C}/TAB are highlighted in black (white writing) and light grey (black writing), respectively. The positions where deletions were included are shown in darker grey (dash).

FIG. 3: Characterisation of H_{C}/TAB. (a) SDS-PAGE analysis of purified H_{C}/TAB, and compared to H_{C}/A and H_{C}/B controls. (b) Western-blot analysis using a poly-Histidine probe, same samples as in (a). ‘M’ denotes the molecular weight markers.

FIG. 4: X-ray crystal structure of the binding domain of TriRecABTox in complex with SV2C, human synaptotagminI and GD1a. (a) Ribbon representation of H_{C}/TAB, with SV2C, hSyt1 and GD1a. (b-d) Example of 2Fₒ-F_{c} electron density map (mesh) at 2σ around the SV2C receptor binding site (b), GD1a (c) and hSyt1 (d).

FIG. 5: Binding to SV2 receptor. (a) Superposition of the crystal structure of H_{C}/TAB and H_{C}/A (PDB 4JRA) in complex with hSV2C. (b) Superposition of the crystal structure of H_{C}/TAB and H_{C}/A (PDB 5JLV) in complex with glycosylated hSV2. Residues involved in binding (Benoit et al., 2014) are shown as sticks, and labelled according to the corresponding H_{C}/A position.

FIG. 6: Binding to synaptotagmin. Superposition of the crystal structure of H_{C}/TAB and H_{C}/B (PDB 4KBB) in complex with human Syt1 and rat Syt2, respectively. Residues involved in binding (Jin et al., 2006; Chai et al., 2006) are shown as sticks, and labelled according to the corresponding H_{C}/B position.

FIG. 7: Binding to GD1a. Superposition of the crystal structure of H_{C}/TAB and H_{C}/B (PDB 4KBB) in complex with GD1a, (dark and light grey, respectively). Residues involved in binding (Berntsson et al., 2013) are shown as sticks, and labelled according to the corresponding H_{C}/B position.

FIG. 8: Characterisation of BoNT/TAB. (a) SDS-PAGE analysis of purified BoNT/TAB, with H_{C}/A and H_{C}/B controls. (b-d) Western-blot analysis using a poly-Histidine probe (b); H_{C}/A (c) and H_{C}/B (d) anti-sera. Same samples as in (a), ‘M’ denotes the molecular weight markers.

FIG. 9: Activation of BoNT/TAB. (a) Schematic representation of the BoNT/TAB construct describing the functional domain organisation. The engineered protease activation site is shown as a dashed black line. The natural disulphide bridge between the light and heavy chains is represented as a plain black line (b) SDS-PAGE analysis of the BoNT/TAB activation assay. Non-reduced (NR) and reduced (R) non-activated BoNT/TAB (left), and Factor Xa-activated BoNT/TAB (right), respectively. The fragments of interest are annotated; ‘M’ denotes the molecular weight markers.

FIG. 10: Extended use of H_{C}/TAB. (a) Schematic representation of potential functional BoNT derivatives associated with H_{C}/TAB. The constructs would consist of the functional BoNT domains from any serotypes or subtypes (‘n’). A protease activation site (dashed black line) should also be included. (b) Schematic representation of potential construct that uses H_{C}/TAB for the transport of cargo protein to the surface of neuronal cells.

FIG. 11: Purification of H_{C}/TAB. (a) Chromatograph (A₂₈₀ trace) from the affinity chromatography purification using a 5 ml HisTrap FF column. (b) Chromatograph (A₂₈₀ trace) from the size exclusion purification using a Superdex200 column. The stages of the purification process and the fractions with H_{C}/TAB are highlighted.

FIG. 12: Crystals of H_{C}/TAB in complex with SV2C, hSyt1 and GD1a. (a) Crystal grown in 20% v/v polyethylene glycol 6000, 0.1 M Citrate pH 5.0. (b) Crystal mounted on a cryo-loop for data collection at Diamond I04-1 station. (c) X-ray diffraction pattern of the crystal.

FIG. 13: Purification of BoNT/TAB. (a) Chromatograph (A₂₈₀ trace) from the affinity chromatography purification using a 5 ml HisTrap FF column. (b) Chromatograph (A₂₈₀ trace) from the size exclusion purification using a Superdex200 column. The stages of the purification process and the fractions with BoNT/TAB are highlighted.

FIG. 14: X-ray crystal structure of the binding domain of H_{C}/TAB in complex with SV2C, human synaptotagmin1 and GD1a. (a) and (b) Temperature facture analysis of the crystal structures of H_{C}/TAB (a) and H_{C}/TAB2.1 (b)-Putty radius representation, where the radius is proportional to the B-factors. Loop ‘360’ is indicated, with positions 360 and 362 highlighted in black. (c) X-ray crystal structure of the complex H_{C}/TAB2.1, with SV2C, hSyt1 and GD1a (stick representation). (d) Same as (c) with the lipid binding loop labelled and hydrophobic residues shown as sticks.

FIG. 15: Purification of H_{C}/TAB2.1. (a) Chromatograph (A280 trace) from the affinity chromatography purification using a 5 ml HisTrap FF column. (b) Chromatograph (A280 trace) from the gel filtration using a Superdex200 column. Fractions with H_{C}/TAB2.1 are indicated. (c) and (d) Characterisation of H_{C}/TAB2.1. SDS-PAGE analysis of fractions from purified H_{C}/TAB2.1, from the affinity chromatography in (c) and gel filtration (d); first lanes on the left show the molecular weight markers. Band corresponding to H_{C}/TAB2.1 is indicated.

FIG. 16: Purification of H_{C}/TAB2.1.1 and H_{C}/TAB2.1.3. (a) and (b) Chromatographs (A280 trace) from the affinity chromatography purification and gel filtration of H_{C}/TAB2.1.1, respectively. Fractions with H_{C}/TAB2.1.1 are indicated. (c) and (d) Chromatographs (A280 trace) from the affinity chromatography purification and gel filtration of H_{C}/TAB2.1.3, respectively. Fractions with H_{C}/TAB2.1.3 are indicated. (d) Characterisation of H_{C}/TAB2.1.1 and H_{C}/TAB2.1.3. SDS-PAGE analysis of the purified samples; first lane on the left shows the molecular weight markers. Bands corresponding to H_{C}/TAB2.1.1 and H_{C}/TAB2.1.3 are indicated

FIG. 17: Figure X4: Purification of BoNT/TAB2.1.3. (a) and (b) SDS-PAGE analysis of fractions from BoNT/TAB2.1.3 purification. Fractions from the affinity chromatography (a) and gel filtration (b); first lanes on the left show the molecular weight markers. Band corresponding to BoNT/TAB2.1.3 is indicated. (c) SDS-PAGE analysis of the purified BoNT/TAB2.1.3 sample. In lane 1: sample before thrombin activation, in lane 2: final activated sample (post-thrombin treatment). Bands corresponding to the full-length (single-chain), HC and LC are indicated. Lane on the right shows the molecular weight markers. (d) Chromatograph (A280 trace) from the final gel filtration (post-thrombin cleavage) using a Superdex200 column. Fractions with BoNT/TAB2.1.3 are indicated.

### DEFINITIONS

As used herein, the term Botulinum neurotoxin “BoNT” encompasses any polypeptide or fragment from a Botulinum neurotoxin. The term BoNT may refer to a full-length BoNT. The term BoNT may refer to a fragment of the BoNT that can execute the overall cellular mechanism whereby a BoNT enters a neuron and inhibits neurotransmitter release. The term BoNT may simply refer to a fragment of the BoNT, without requiring the fragment to have any specific function or activity.

As used herein, the term “translocation domain” or “H_{N}” means a BoNT domain that can execute the translocation step of the intoxication process that mediates BoNT light chain translocation. Thus, an H_{N} facilitates the movement of a BoNT light chain across a membrane into the cytoplasm of a cell.

As used herein, the term “binding domain” is synonymous with “H_{C} domain” and means any naturally occurring BoNT receptor binding domain that can execute the cell binding step of the intoxication process, including, e.g., the binding of the BoNT to a BoNT-specific receptor system located on the plasma membrane surface of a target cell.

In the present disclosure, the terms “nucleic acid” and “gene” are used interchangeably to describe a nucleotide sequence, or a polynucleotide, encoding for a polypeptide.

### DETAILED DESCRIPTION

As specified above in the background section, a BoNT comprises a light chain (LC), linked by a single disulphide bridge to the heavy chain (HC). The Heavy chain (HC) holds two of the functional domains, with the N-terminal translocation domain (H_{N}) and the C-terminal binding domain (H_{C}), while LC is responsible for intracellular catalytic activity. The H_{C} thus comprises the receptor binding domains which are able to specifically and irreversibly bind to the specific receptors expressed on susceptible neurons, whereas the H_{N} forms a channel that allows the attached LC to translocate from endosomal-like membrane vesicles into the cytosol. Different BoNT serotypes have different sets of receptor binding sites on the H_{C}, typically two receptor binding sites. The inventors have made use of this knowledge in engineering a novel BoNT H_{C} binding domain (H_{C}/TAB) comprising binding sites for three different receptors.

The inventors have accomplished this by engineering a H_{C}/TAB domain comprising:
a) a synaptotagmin (Syt) receptor binding site, and
b) a synaptic associated vesicle 2 (SV2) receptor binding site, and
c) a ganglioside (Gang) receptor binding site.

The structure of the engineered H_{C}/TAB domain allows the H_{C}/TAB to synergistically bind to a synaptotagmin (Syt) receptor, a synaptic associated vesicle 2 (SV2) receptor and a ganglioside (Gang) receptor. Thus a synergistic binding to three receptors on the neuron cell is accomplished, causing the novel H_{C}/TAB domain to have enhanced affinity as compared to other BoNT H_{C} domains. Thus an overall binding to neurons is improved and consequently the efficacy of the toxin is improved.

The H_{C} further comprises an N-terminal end (H_{CN}) and a C-terminal end (H_{CC}). A key feature of the present invention is the structure of the H_{CC} end of the H_{C}/TAB, which is where the receptor binding domains are located in BoNT.

In one embodiment of the H_{C}/TAB, the H_{CC} end is composed interchangeably of sequences from the BoNT serotype A (BoNT/A) and BoNT serotype B (BoNT/B). By engineering this interchangeable structure, the inventors have been able to optimize a synergistic binding to all three receptors.

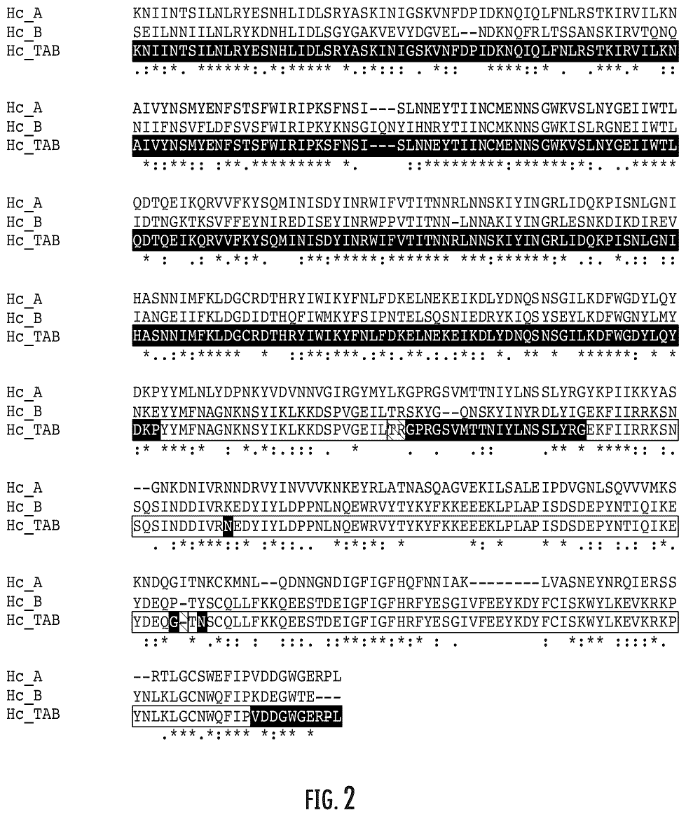

In a further embodiment of the invention, the H_{CC} end is composed according to a sequence A₁B₁A₂B₂A₃, where A indicate a sequence from BoNT/A, and B indicate a sequence from BoNT/B, see FIG. 2. This further optimizes the structure of the H_{C}/TAB, in allowing the three receptor binding domains to at least synergistically bind to all three said receptors, possibly even simultaneously. The inventors have shown that simultaneous binding to all three receptors occurs in vitro with this A₁B₁A₂B₂A₃ sequence. The engineered A₁B₁A₂B₂A₃ sequence according to this particular embodiment is described in SEQ. ID. No. 1

In order to further optimize the H_{C}/TAB according to the above, mutations and deletions have been introduced to create stable intramolecular interfaces, see FIG. 2. In SEQ. ID. No. 1, substitutions have been made in positions 306, 360 and 362, and deletions have been made, compared to the original sequence, between positions 265/266 and 360/361. However, the skilled person will appreciate that mutations and/or deletions for an amino acid at a position +1, +2, +3, +4, +5, or −1, −2, −3, −4 or −5 from the above specified positions may have the same effect. Thus, any such modification at a position of +/−5 amino acids from the specified amino acid positions falls within the scope of the present disclosure.

According to specific embodiments above, and all of the examples below, the ganglioside receptor binding site originates from BoNT/B, but it is conceivable that it may originate from any Gang receptor-binding BoNT serotype and their subtypes, such as the BoNT serotypes A, B, C, D, DC, E, En, F, G or X, or subtypes thereof, since all of the serotypes have a ganglioside receptor binding site.

According to a preferred embodiment of the present invention, the sequences forming the Gang receptor binding site are located in B₂.

The SV2 receptor binding domain normally may originate from any SV2 receptor binding BoNT serotype and their subtypes, and in particular from BoNT serotypes A, D, E and F. In the specific embodiments above and all of the examples below, the SV2 receptor binding domain originates from BoNT/A, but as the skilled person will appreciate, any serotype comprising a SV2 receptor binding domain may be used as the origin for said domain, in accordance with the purpose and intended use of the H_{C}/TAB according to the appended claims.

Part of the SV2 receptor binding domain is present in the H_{CN} end. Thus, as a consequence the H_{CN} sequence may originate from any of the SV2-receptor binding BoNT serotypes and their subtypes. In the specific embodiments above and all of the examples below, the H_{CN} end originates from BoNT/A. However, as the skilled person will appreciate, as long as the SV2 receptor binding domain is functional, the H_{CN} sequence may also originate from any of BoNT serotypes C, D, E, F or G.

Furthermore, according to a preferred embodiment of the present invention, the sequences forming the SV2 receptor binding site are located in H_{CN} and in A₁ and A₃ in the H_{CC}.

The Syt receptor binding site may originate from any Syt receptor binding BoNT serotype and their subtypes. In particular, the Syt receptor binding site may originate from BoNT serotypes B, chimera DC or G. According to a preferred embodiment of the present invention, the sequences forming the Syt receptor binding site are located in B₁ and B₂.

The present invention also provides for a polypeptide comprising the H_{C}/TAB according to the above. The polypeptide may thus comprise any other protein, polypeptide, amino acid sequence or fluorescence probe, being coupled to the H_{C}/TAB either directly or via a linker. Hereinafter, a protein, polypeptide or amino acid sequence to be coupled to the H_{C}/TAB is referred to as “protein”.

According to one preferred embodiment, the polypeptide is a recombinant BoNT polypeptide (BoNT/TAB) further comprising a H_{N} and a LC, as well as an exoprotease site positioned between the LC and H_{N} in the polypeptide sequence.

The exoprotease site enables the single-chain polypeptide to be cleaved into a di chain molecule, causing the molecule to become an active toxin. According to an embodiment of the invention, the exoprotease site is a Factor Xa site, although this is not a limiting feature of the polypeptide according to the invention.

According to one embodiment, the BoNT/TAB in its active form is according to the SEQ. ID. No. 5. According to another embodiment, the BoNT/TAB in its active form is according to any of the sequences of SEQ. ID. No's 6, 8, 10 or 12. Preferably, the BoNT/TAB in its active form is according to SEQ. ID. No. 12.

Both the H_{N} and the LC may, respectively and independently, originate from any of the BoNT serotypes A, B, C, D, DC, E, En, F, G or X and their subtypes, as well as BoNT-like polypeptides. New proteins resembling BoNT, i.e. with a similar domain architecture and varying degree of sequence identity, but produced by other organisms than C.-botulinum, are emerging. Thus, the skilled person will be able to choose a H_{N} and/or a LC from any of the BoNT serotypes, their subtypes, or BoNT-like polypeptides.

The mutations and deletions that are introduced in the H_{C}/TAB as specified above, further ensure that an engineered BoNT/TAB may be produced as a soluble protein with the correct structure and required activity.

A polypeptide according to the above is preferably produced recombinantly as the H_{C}/TAB needs to be produced recombinantly.

Thus, the present disclosure also provides for isolated and/or recombinant nucleic acids encoding any of the H_{C}/TAB or polypeptides according to the above. The nucleic acids encoding the H_{C}/TAB or polypeptides of the present disclosure may be DNA or RNA, double-stranded or single stranded. In certain aspects, the subject nucleic acids encoding the isolated polypeptide fragments are further understood to include nucleic acids encoding polypeptides that are variants of any of the H_{C}/TAB or polypeptides described herein. Variant nucleotide sequences include sequences that differ by one or more nucleotide substitutions, additions or deletions, such as allelic variants.

The present invention also provides for a vector comprising a nucleic acid sequence encoding the H_{C}/TAB according to the above. The vector may further comprise a nucleic acid sequence encoding any other protein or probe that is to be recombinantly produced together with the H_{C}/TAB, so as to obtain said protein or probe coupled to the H_{C}/TAB in one polypeptide. The vector is preferably an expression vector. The vector may comprise a promoter operably linked to the nucleic acid. A variety of promoters can be used for expression of the polypeptides described herein, and are known to the person skilled in the technical field.

An expression vector comprising the nucleic acid can be transferred to a host cell by conventional techniques (e.g., electroporation, liposomal transfection, and calcium phosphate precipitation) and the transfected cells are then cultured by conventional techniques to produce the polypeptides described herein. In some embodiments, the expression of the polypeptides described herein is regulated by a constitutive, an inducible or a tissue-specific promoter.

The polypeptides may be produced in any cells, eukaryotic or prokaryotic, or in yeast. The polypeptides according to the invention may further be produced in a cell free system. The skilled person will be readily able to apply the expression system of choice to that person. The expression system used for producing the polypeptides of the invention are not limiting to the scope of the invention.

Purification and modification of recombinant proteins is well known in the art such that the design of the polyprotein precursor could include a number of embodiments readily appreciated by a skilled worker.

The protein to be included in the polypeptide may be any protein of interest to be transported to a neuronal cell, and/or internalized into a neuronal cell.

It may be advantageous to comprise a H_{N} according to the above in the polypeptide together with the H_{C}/TAB, and replace the LC with the protein of interest, if an internalization of the protein is desired, as the H_{N} then will provide a channel allowing the protein to translocate into the neuronal cell. It may be advantageous to couple the protein of interest directly to the H_{C}/TAB if the neuronal cell surface is the target for the protein. Thus, the following combinations may be obtained, depending on the aimed delivery:
i) Protein—H_{C}/TAB
ii) Protein—H_{N}-H_{C}/TAB
iii) Protein—L_{C}-H_{N}-H_{C}/TAB

By coupling a cargo protein to the H_{C}/TAB, according to i) above, the cargo protein may be targeted to the neuronal surface. Some internalisation via regular cell surface recycling processes would probably occur, but the neuronal surface would be the main target of such an approach.

By coupling a cargo protein to a H_{N} coupled to the H_{C}/TAB according to ii) above, or to the BoNT/TAB according to iii) above, said cargo proteins may be more effectively transported inside neurons using the toxin translocation system. Once the BoNT toxin has been internalized in the neuron cell in the vesicles, as described in the background, the acidic endosomal environment in the vesicle causes a conformational change that allows translocation of LC from the vesicle into the cytosol of the cell. Thus, said toxin translocation system which is the mechanism for translocating the LC of BoNT from the internalized vesicle into the cytosol, may be used to translocate the above mentioned cargo protein into the cytosol of the neuron cell, by use of the BoNT/TAB. A cargo protein may be coupled to the H_{N} instead of the LC, with an exoprotease site positioned between the cargo protein and H_{N} as disclosed above, or a cargo protein may be coupled to the LC. Both variants will enable a transportation of the cargo protein into the cytosol of the neuronal cell. Thus, both the H_{C}/TAB and the BoNT/TAB may be used as vehicles for transporting any protein to and/or into a neuron. This also provides for the possibility of using the H_{C}/TAB and/or the BoNT/TAB in a pharmacological test to investigate the role of a protein in for instance a synaptic process.

The cargo protein may for instance be any protein tag, such as affinity or fluorescent tags or probes. Thus, any corresponding nucleic acid to such a protein tag may be included in the vector disclosed above. The skilled person will be able to use standard cloning methods in order to comprise any gene of interest in the vector, as well as standard protocols for the protein expression.

The binding domain of BoNT and the cargo protein could be expressed separately with a sortase system that allow their recombination post-translationally. The transpeptidase activity of sortase may thus be used as a tool to produce fusion proteins in vitro and is well within the knowledge of a skilled person within this technical field. In short, a recognition motif (LPXTG) (SEQ ID NO: 15) is added to the C-terminus of a protein of interest while an oligo-glycine motif is added to the N-terminus of the second protein to be ligated. Upon addition of sortase to the protein mixture, the two peptides are covalently linked through a native peptide bond. This method may be used to produce a polypeptide according to the present invention. In the present case, this would mean that the recognition motif is added to the C-terminus of the protein of interest, and the oligo-glycine moif is added to the N-terminus of the H_{C}/TAB or BoNT/TAB.

Additionally, the H_{C}/TAB and/or the BoNT/TAB may be used in a therapeutic method or cosmetic method. Typically, the use of H_{C}/TAB and/or the BoNT/TAB may be very similar to the uses that are already in place for BoNT/A and/or BoNT/B products. These include methods and treatments wherein the purpose of the method and treatment is to dampen and/or inactivate muscles.

The H_{C}/TAB according to the invention enables injections of a BoNT/TAB having a higher affinity to the cell and consequently a higher efficiency. Thus, lower doses are required and a longer duration of action is possible. Therefore, a smaller amount of BoNT/TAB as compared to BoNT/A or BoNT/B, may be injected for the same effect, which decreases adverse effects as less BoNT/TAB will spread from the site of injection. With a higher efficiency, stronger and more efficient binding, and lower dose required, there are less redundant BoNT/TAB available to spread to beyond the injection site. Furthermore, the BoNT could be administered less often with sustained effect, which would also minimize the risk of an immune response and adverse reactions as a consequence thereof.

Typical medical conditions that may be treated and/or prevented with the H_{C}/TAB and/or the BoNT/TAB according to the above are disorders chosen from the group comprising neuromuscular disorders, conditions involving the release of acetylcholine, and spastic muscle disorders. More specifically is may relate to disorders chosen from the group comprising of spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, ties, tremors, bruxism, anal fissure, achalasia, dysphagia and other muscle tone disorders and other disorders characterized by involuntary movements of muscle groups, lacrimation, hyperhydrosis, excessive salivation, excessive gastrointestinal secretions, secretary disorders, pain from muscle spasms, headache pain, sports injuries, and depression.

With regards to cosmetic methods, the H_{C}/TAB and/or the BoNT/TAB may preferably be used to prevent and/or treat wrinkles, brow furrows or unwanted lines, in order to reduce said wrinkles, furrows and lines.

The H_{C}/TAB and/or the BoNT/TAB according to the above may be formulated in any suitable pharmaceutical or cosmetic composition. The pharmaceutical composition comprising the H_{C}/TAB and/or the BoNT/TAB may further comprise pharmaceutically acceptable excipients, carriers or other additives. The cosmetic composition comprising the H_{C}/TAB and/or the BoNT/TAB may further comprise cosmetically acceptable excipients, carriers or other additives.

The administration of the pharmaceutical or cosmetic composition may be via injection, wherein the injection is administered at the site of the body where unwanted neuronal activity is present. Typically, compositions for administration by injection are solutions in sterile isotonic aqueous buffer. Where necessary, the composition can also include a solubilizing agent and a local anesthetic to ease pain at the site of the injection.

Furthermore, the pharmaceutical or cosmetic composition may be comprised in a kit with directions for therapeutic administration of the composition. In such a kit, the ingredients of the composition may be supplied either separately or mixed together in unit dosage form, for example, as a dry lyophilized powder or water free concentrate in a hermetically sealed container such as an ampoule or sachette indicating the quantity of active agent. The composition may be administered by infusion, and can in that case be dispensed with an infusion bottle containing sterile pharmaceutical grade water or saline. Where the composition is administered by injection, an ampoule of sterile water for injection or saline can be provided so that the ingredients can be mixed prior to administration. A composition for systemic administration may be a liquid, e.g., sterile saline, lactated Ringer's or Hank's solution. In addition, the composition can be in solid forms and re-dissolved or suspended immediately prior to use. Lyophilized forms are also contemplated. The composition can be contained within a lipid particle or vesicle, such as a liposome or microcrystal, which is also suitable for parenteral administration.

Thus, the inventors have developed an engineered BoNT biohybrid adapted to simultaneously bind to all three of the SV2C receptor, the synaptotagmin receptor and the ganglioside receptor. Thereby, a BoNT biohybrid is provided that has a higher potency, efficacy and duration than the BoNT polypeptides of the prior art. Use of the present biohybrid thereby enables administration of lower doses of the toxin than according to the prior art, while maintaining the same effect. Furthermore, use of the present biohybrid enables less frequent administrations than for the BoNT's previously used. Thus, a treatment of a patient with the BoNT biohybrid of the present invention will be more comfortable in that administration does not have to occur as often as in the prior art.

### EXPERIMENTAL SECTION

### Material and Methods

Constructs. The cDNA encoding H_{C} and full-length (inactive) TriRecABTox (H_{C}/TAB and BoNT/TAB, respectively) were codon-optimised for E. coli expression (see supplementary information for DNA sequence), synthesised and cloned into a pET-28a(+) vector with a N-terminal 6×His-tag (GenScript, NJ, USA). The TriRecABTox construct used in our study has three mutations at the catalytic site to avert any safety concerns (E224Q/R363A/Y366F) (Rossetto et al, 2001; Binz et al, 2002). The BoNT/TAB gene encodes for 1311 amino acids, and the H_{C}/TAB gene corresponds to residues [875-1311].

Protein expression and purification. Plasmids carrying the gene of interest were transformed into E. coli BL21 (DE3) cells (New England Biolabs, USA). A similar protocol was used for both proteins. Expressions were carried out by growing cells in terrific broth medium with 50 μg/ml kanamycin at 37° C. for approximately 3 hours and then induced with a 1 mM final concentration of IPTG, and left overnight at 18° C., in a LEX system (Epyphite3, Canada). Cells were harvested and stored at −80° C. Cell lysis for protein extraction was performed with an Emulsiflex-C3 (Avestin, Germany) at 20 kPsi in 25 mM HEPES pH 7.2 with 200 mM NaCl, 25 mM imidazole and 5% (v/v) glycerol. Cell debris were spun down via ultra-centrifugation at 4° C., 267,000 g for 45 min. The protein was first purified by affinity chromatography: the supernatant was loaded onto a 5 ml HisTrap FF column (GE Healthcare, Sweden), washed with 25 mM HEPES pH 7.2, 200 mM NaCl, 25 mM imidazole and 5% (v/v) glycerol, and the protein eluted with 25 mM HEPES PH 7.2, 200 mM NaCl, 250 mM imidazole and 5% (v/v) glycerol. The sample was then dialysed against 25 mM HEPES pH 7.2, 200 mM NaCl, and 5% (v/v) glycerol overnight, before a final size exclusion purification step using a Superdex200 column in a similar buffer (GE Healthcare, Sweden). H_{C}/TAB was kept at 4.5 mg/ml, and BoNT/TAB at 7.3 mg/ml, in 25 mM HEPES pH 7.2 with 200 mM NaCl, 0.025 mM TCEP and 5% glycerol.

Protein characterisation. Protein samples were analysed by gel electrophoresis using NuPAGE 4-12% Bis-Tris gels, and Western blots performed on PVDF membranes (ThermoFisher, Sweden). Primary antibodies against H_{C}/A and H_{C}/B were prepared in-house (raised in rabbit) and probed with an anti-rabbit IgG-Peroxidase antibody (catalogue #SAB3700852, Sigma, Sweden). The poly-histidine tag was probed using an HRP-conjugated monoclonal antibody (ADI.1.10, catalogue #MAI-80218, ThermoFisher, Sweden). TMB substrate (Promega, Sweden) was used for detection. In-house controls purified similarly to H_{C}/TAB and consisting of His-tagged H_{C}/A and H_{C}/B were included for comparison.

Activation of BoNT/TAB. The full-length (inactive) TriRecABTox was designed with a Factor Xa cleavage site (IEGR) between the light and heavy chains for activation into a di-chain form. Activation was performed by incubating 100 μg of BoNT/TAB with 2 μg. of Factor Xa (New England BioLabs, USA) overnight at 4° C. Results of the activation was analysed by gel electrophoresis (as above).

Cloning, expression and purification of SV2C-L4. The interacting part of the fourth luminal domain of synaptic vesicle glycoprotein 2C (SV2C-L4, residues 474-567 Uniprot ID Q496J9) was amplified from cDNA and cloned into a pNIC28-Bsa4 (N-terminal His6 tag with TEV site) vector using LIC cloning. SV2CL4 was expressed in E. coli BL21 (DE3) (New England Biolabs, USA) using a protocol similar to the one described above. His-tagged SV2C-L4 was purified by affinity chromatography on a 2 ml HisTrap HP column (GE Healthcare, Sweden), washed with 20 mM HEPES, pH 7.5, 500 mM NaCl, 10% (v/v) glycerol, 50 mM Imidazole, and 0.5 mM TCEP. The protein eluted with 20 mM HEPES, pH 7.5, 500 mM NaCl, 10% (v/v) glycerol, 500 mM Imidazole, and 0.5 mM TCEP. SV2CL4 was then purified further by size exclusion using a Superdex 75 Hiload 16/60 column (GE Healthcare, Sweden) in 20 mM HEPES, pH 7.5, 300 mM NaCl, 10% (v/v) glycerol, and 0.5 mM TCEP.

X-ray crystallography. Samples for crystallisation were prepared by pre-incubation for 15 min at room temperature of H_{C}/TAB at 3.6 mg/ml, with SV2C-L4 at 1 mg/ml (recombinant human SV2C extracellular loop-4 [residues 475-565], 1 mM hSytI peptide (GEGKEDAFSKLKEKFMNELHK (SEQ ID NO: 14), synthesised by GenScript, USA) and 4 mM GD1a oligosaccharide (Elicityl, France).

Crystals were grown with 200 nl of sample mixed with 100 nl of reservoir solution consisting of 20% v/v polyethylene glycol 6000, 0.1 M Citrate pH 5.0 (JCSG-plus screen B9, Molecular Dimensions, United Kingdom) using a sitting drop set-up and incubated at 21° C. Crystals appeared within 2 weeks and were transferred to a cryo-loop and frozen in liquid nitrogen.
Diffraction data were collected at station I04-1 of the Diamond Light Source (Didcot, UK), equipped with a PILATUS-6M detector (Dectris, Switzerland). A complete dataset to 1.5 Å was collected from a single crystal at 100° K. Raw data images were processed and scaled with DIALS (Gildea et al, 2014), and AIMLESS (Evans, 2006) using the CCP4 suite 7.0 (CCP4, 1994).

Molecular replacement was performed with a model prepared from the coordinates of H_{C}/A in complex with SV2C-L4 (PDB code 4JRA) and of H_{C}B in complex with rat SytII and GD1a (PDB code 4KBB) to determine initial phases for structure solution in PHASER (McCoy et al., 2007). The working models were refined using REFMAC5 (Murshudov et al, 2011) and manually adjusted with COOT (Emsley et al., 2010). Water molecules were added at positions where Fo-Fc electron density peaks exceeded 3σ, and potential hydrogen bonds could be made. Validation was performed with MOLPROBITY (Chen et al., 2010). Ramachandran statistics show that 97.0% of all residues are in the most favoured region, with a single outlier in the disallowed region. Crystallographic data statistics are summarized in Table 1. Figures were drawn with PyMOL (Schrodinger, LLC, USA).

### Results

### Design of TriRecABTox: an Engineered Botulinum Toxin With Three-Receptor Binding Sites.

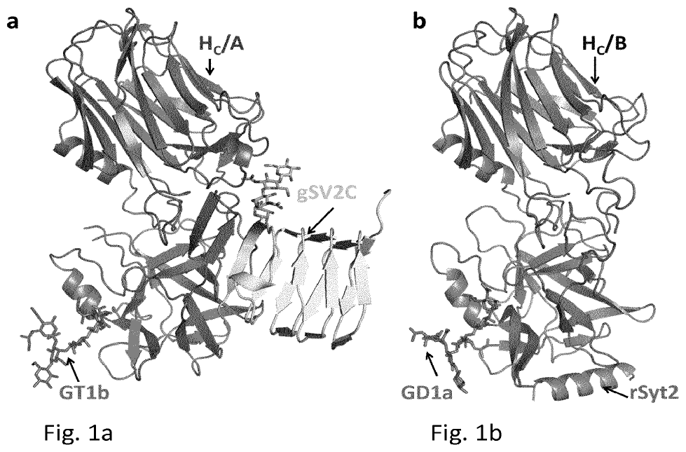

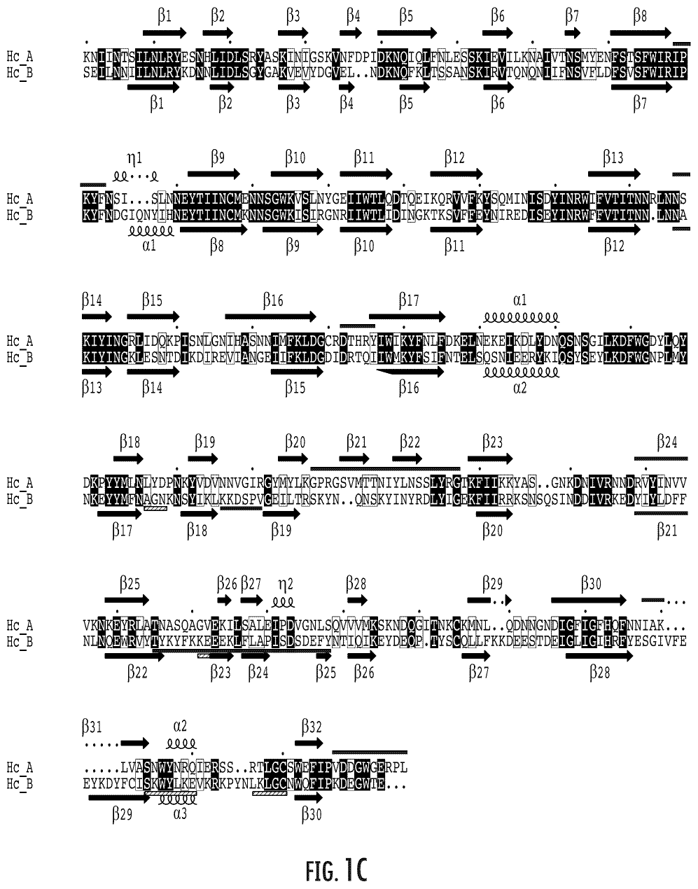

In order to materialise the concept of a three-receptor toxin, the inventors first analysed the structural information available on the BoNT/A and /B molecular interactions with their receptors. Recent work by Yao et al. (2016) and Benoit et al. (2014) provided the X-ray crystal structures of the receptor-binding domain of BoNT/A in complex with SV2C with (PDB 5JLV) and without post-translation modification (PDB 4JRA), respectively. The luminal domain of SV2C (loop4) forms a quadrilateral β-helix that associates with H_{C}/A mainly through backbone-to-backbone interactions with an opened β-strand at the interface of the two subdomains, while the N-glycan of SV2C extends towards H_{CN} (FIG. 1). Together these structures demonstrated a common binding mode to the two SV2 forms that should also extend to glycosylated SV2A and SV2B (Yao et al., 2016). These studies highlighted the key residues and multiple sites involved in the toxin-SV2 interaction that should thus be kept in the design of TriRecABTox (FIG. 1). These included segments [949-953], [1062-1066], [1138-1157] and [1287-1296] of BoNT/A. Residue numbers are based on sequence of BoNT/AI (Uniprot-P10845).

Several crystal structures of BoNT/B in complex with synaptotagmin have also been described and helped define the toxin's interaction with its receptor (Chai et al., 2006; Jin et al., 2006; Berntsson et al., 2013) (FIG. 1). Upon binding, the Syt peptide takes on a short helical structure that binds along a groove on the distal tip of the C-terminal subdomain, directly involving segments [1113-1118] and [1183-1205] of BoNT/B. Residue numbers are based on sequence of BoNT/B1 (Uniprot-P10844). These regions were therefore considered essential to include in the TriRecABTox construct.

Additionally, the crystal structures BoNT/A and /Bin complex with their ganglioside receptor (Stenmark et al., 2008; Hamark et al., 2017; Berntsson et al., 2013) provided a detailed description of the carbohydrate binding site for each serotype. The site is highly conserved across the botulinum neurotoxin family and consists of a shallow pocket on the H_{CC} subdomain (FIG. 1) composed of the central SxWY motif (1264-1267 in/A; 1260-1263 in/B), and the surrounding loop regions. Noticeably, this pocket is adjacent to the Syt receptor binding site in BoNT/B, separated by loop [1244-1253], however no allosteric effect was reported upon simultaneous binding of the two receptors (Bertnsson et al., 2013). In the interest of minimising any structural alteration to the Syt receptor binding site, it was deemed more suitable to incorporate the ganglioside receptor-binding site of BoNT/B, rather than BoNT/A, in the design of TriRecABTox.

After identification of the components from the two serotypes that are essential for binding to the three different receptors, further structural analysis was performed to integrate them into a single molecule. To this extent, the primary sequences of BoNT/A (Uniprot P10845) and BoNT/B (Uniprot P10844) were aligned with ClustalO (Sievers et al., 2011), and the three-dimensional structures of their binding domain superposed (FIG. 1). The two serotypes share an overall sequence identity of 40%, however the similarity drops to 34% for the C-terminal subdomain of H_{C}, the main region responsible for receptor recognition. The core fold of the binding domain is conserved across all clostridial neurotoxins (Swaminathan, 2011; Rummel et al., 2011), but with noticeable variation in the length of the connecting loops. It was therefore important to also take into account the secondary structures (FIG. 1), so as to keep the main architecture of the domain intact. The template for the newly designed molecule consequently appeared as multiple alternations between BoNT/A and /B elements, creating novel non-natural intra-molecular interfaces that may not be compatible. Inspection of the superposed crystal structures of H_{C}/A and H_{C}/B allowed the inventors to optimise the design by correcting potential clashes, either by single amino substitutions or deletions in key locations (FIG. 2). In particular, the side chain of every residue within the conflicting areas was reviewed, resulting in three substitutions from BoNT/B to the equivalent BoNT/A amino acid: N1180, G1234, N1236 (SEQ. ID. No. 3). Additionally, several amino acids were removed (FIG. 2) in order to match the secondary structure elements and compensate the length variations between BoNT/A and /B in the loop regions of the transition interfaces. Deletions have been made between L1139 and G1140, as well as between G1234 and T1235 (referring to SEQ. ID.No. 3), compared to the BoNT/A and BoNT/B sequences (FIG. 2)

The resulting molecule, named TriRecABTox, should be able to bind to the three receptors: SV2, synaptotagmin and gangliosides. Its protein sequence is provided in SEQ. ID. No. 3 (inactive form) and SEQ. ID. No 5 (active form).

### Production and Characterisation of the TriRecABTox Binding Domain.

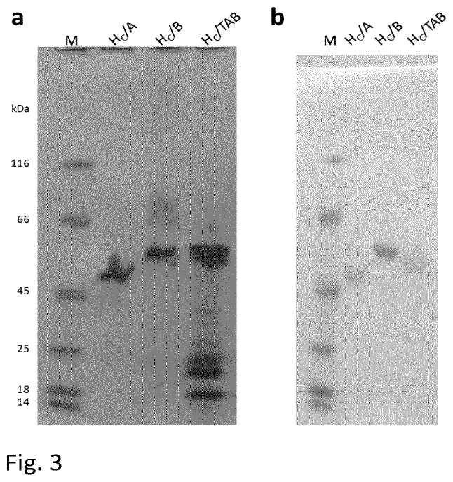

The first step towards the characterisation of TriRecABTox was to recombinantly produce the binding domain (H_{C}/TAB) in order to analyse its biochemical properties. For this purpose, the protein sequence was codon-optimised for expression in E. coli. The resulting gene was cloned into a pET-28a(+) vector so as to include a N-terminal poly-histidine tag and facilitate the protein purification process, details are provided in the methods section. The inventors showed that H_{C}/TAB could be expressed and partially purified (FIG. 3) using affinity chromatography and size exclusion techniques (FIG. 11). The original sample presented some low molecular weight contaminants that likely correspond to residual host cell proteins. Additional purification steps using methods such as ion exchange or hydrophobic interaction chromatography should help obtain a sample of higher purity. Presence of the His-tagged He/TAB was confirmed by Western blot where a single band at the expected size (approximately 53 kDa) was observed (FIG. 3).

### Crystal Structure of the TriRecABTox Binding Domain in Complex With its Three Receptors.

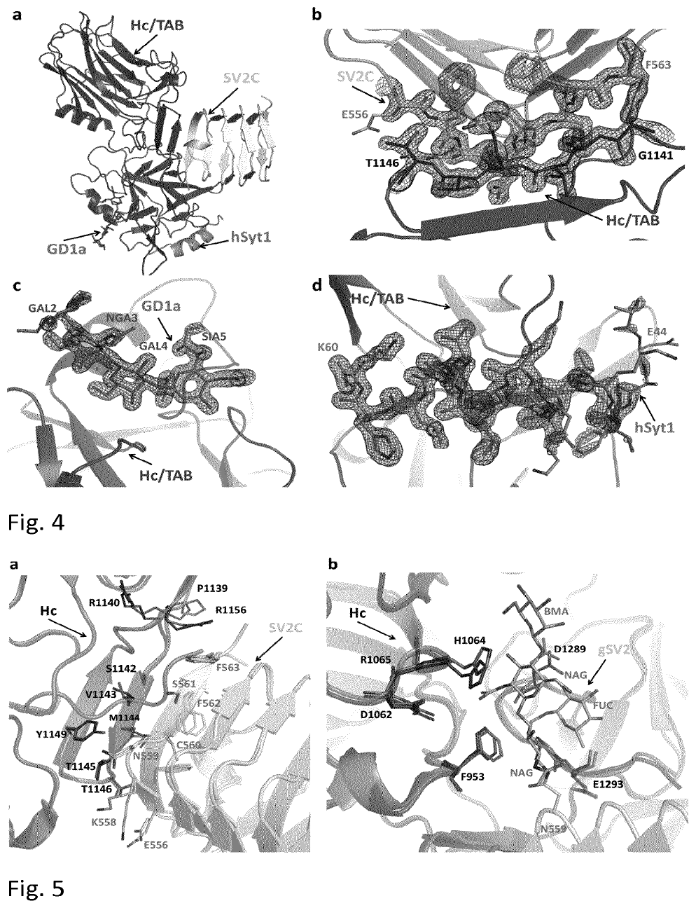

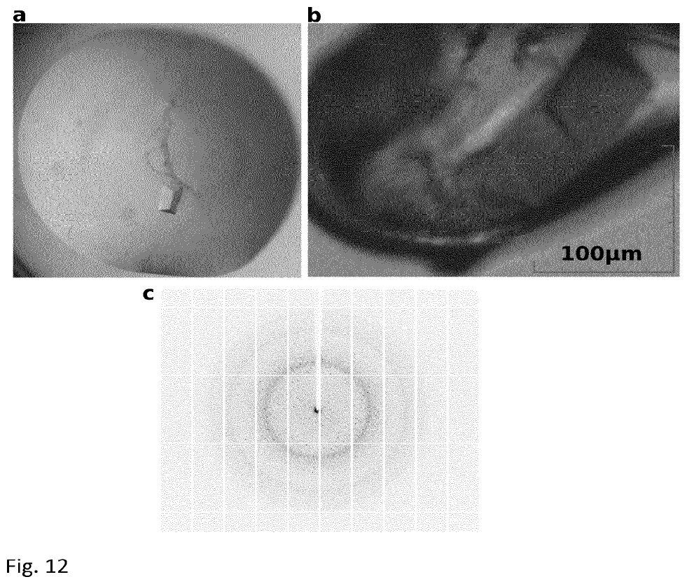

In an effort to evaluate the capacity of H_{C}/TAB to bind to its three receptors, co-crystallisation trials were set up that included H_{C}/TAB with the human SV2C luminal domain [residues 475-565], the human Syt1 peptide [residues 34-53] and the GD1a carbohydrate. Crystals were obtained that diffracted to high resolution (1.5. Å) (FIG. 12) and a complete dataset could be collected (Table 1). The structure was solved by molecular replacement using an input model with all the potential components. The solution confirmed that the crystal structure contained all four elements: H_{C}/TAB bound to it three receptors simultaneously (referred to as H_{C}/TAB-3R) (FIG. 4). This result provides the first experimental evidence that TriRecABTox can achieve its purpose in vitro, and also allowed a complete analysis of the receptor binding mechanism in atomic details. Using the newly determined structural information, we could directly compare the interaction between H_{C}/TAB, H_{C}/A, H_{C}/B, and their respective receptors.

TABLE 1
X-ray crystallography: data collection and refinement statistics

| | H_{C}/TAB-SV2C-hSytl-GD1a complex | |
|---|---|---|
| Data collection | | |
| Space group | P2₁2₁2₁ | |
| Cell dimensions | | |
| a, b, c (Å) | 43.7, 115.9, 141.4 | |
| a, b, g (°) | 90.0, 90.0, 90.0 | |
| Resolution (Å) | 1.5-60.4 (1.51-1.53)* | |
| No. total/unique | 3,583,212/113,806 | |
| reflections | | |
| R_{merge} | 0.119 (1.926)* | |
| Rₚᵢₘ | 0.021 (0.501)* | |
| CC_{1/2} | 1.00 (0.839)* | |
| I/sl | 13.0 (1.1)* | |
| Completeness (%) | 99.9 (97.2)* | |
| Redundancy | 31.5 (15.1)* | |
| Refinement | | |
| R_{work}/R_{free} (%) | 17.4/22.1 | |
| No. atoms | | |
| H_{C}/TAB | 3,682 | 5 |
| SV2C | 761 | |
| hSytl | 143 | |
| GD1a | 56 | |
| Water | 446 | |
| B-factors | | |
| H_{C}/TAB | 27.4 | |
| SV2C | 44.4 | 10 |
| hSytl | 35.8 | |
| GD1a | 36.8 | |
| Water | 40.2 | |
| R.m.s. deviations | | |
| Bond lengths (Å) | 0.009 | |
| Bond angles (°) | 1.37 | 15 |

*Values in parentheses are for highest-resolution shell.

Firstly, the binding domain of the newly designed BoNT/TAB presents the expected fold with its two subdomains: the lectin-like H_{CN} and the β-trefoil fold of H_{CC} (FIG. 4). The multiple new intra-molecular interfaces created did not perturb the overall structure, as illustrated by the low root mean square deviations (rmsd) of 0.69 Å (over 364 Cα) when superposed with H_{C}/A, and of 0.81 Å (over 370 Cα) with H_{C}/B. The complete H_{C}/TAB was modelled [876-1311] except for the N-terminal poly-Histidine tag and loop [1169-1173] that were disordered. The lack of electron density for these parts may be explained by the facts that these regions are not involved in any interaction, and located within solvent-accessible areas of the crystal.

The H_{C}/TAB-3R structure was compared to that of H_{C}/Å in complex with SV2C. The structure of the SV2C luminal domain is identical in both complexes, with an rmsd of 0.483 Å (over 88 Cα). The two structures were aligned in three-dimension based on the H_{C} domains and showed that SV2C is in the same location, as expected from the inventor's design (FIG. 5). In particular, regions from H_{C}/A that had been designated as necessary for SV2 receptor binding and were included in H_{C}/TAB are fully preserved. The interface between H_{C}/A and SV2C was analysed with PISA (Kissinel, 2015) and corresponds to a surface area of 540 A² involving mostly electrostatic interactions where open strands from both proteins form a complementary β-sheet structure (Benoit et al., 2014). The corresponding analysis with H_{C}/TAB shows a surface area with SV2C of 630 Å² and confirmed the binding mechanism with a comparable number of hydrogen bonds. In addition, the inventors also considered the potential binding to glycosylated SV2 by comparing H_{C}/TAB-3R with the H_{C}/A-gSV2C complex (FIG. 5). N-glycosylation of N559 was recently shown to be essential for receptor recognition and is conserved across SV2 isoforms (Yao et al., 2016). Noticeably, the protein-protein interaction between H_{C}/A and SV2C is highly similar with or without glycosylation. The carbohydrate chain extends towards the H_{CN} subdomain. Analysis of the H_{C}/A residues involved in the protein-glycan interaction shows that their position is completely conserved in H_{C}/TAB-3R, thus H_{C}/TAB should be able to recognise the N-glycosylated isoforms of SV2.

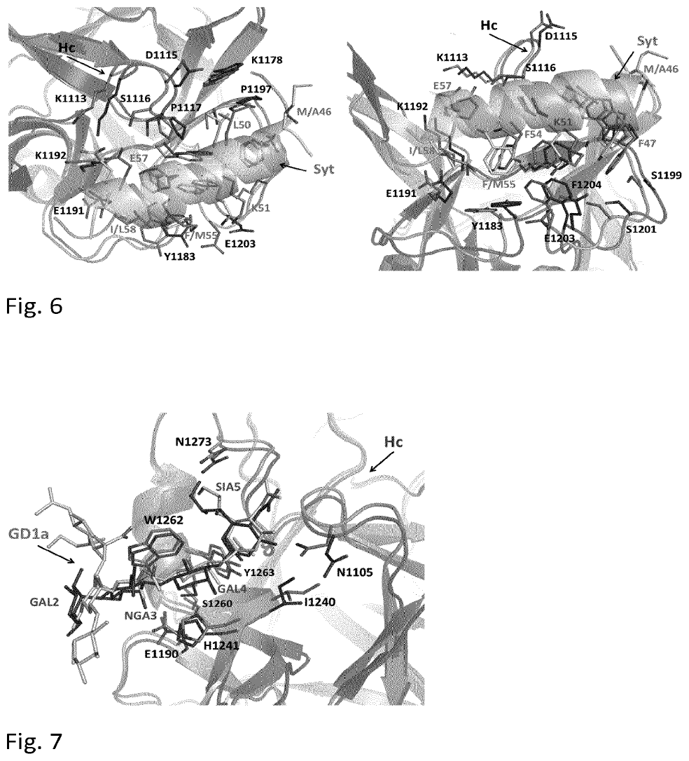

The inventors then compared the H_{C}/TAB-3R structure with that of H_{C}/B in complex with rSyt2. BoNT/B is expected to bind to human synaptotagmin in a similar fashion to its rodent homologues, albeit with varying affinities (Tao et al., 2017). In the crystal structure presented here, hSyt1 also takes on an α-helical arrangement that sits within the same binding groove as rSyt2 in H_{C}/B (FIG. 6). Superposition of hSyt1 with rSyt2 bound to their respective H_{C} domains confirms the conserved peptide configuration with an rmsd of 0.560 Å (over 13 Cα). Additionally the receptor-binding pocket is completely preserved in H_{C}/TAB, with all residues involved in the binding presenting a similar configuration in both structures (FIG. 6). This was confirmed with a PISA analysis where an interface of 861 Å² was calculated for the H_{C}/TAB:hSyt1 interaction that also includes eleven electrostatic bonds, and which is comparable to the 712 Å² H_{C}/B:rSyt2 interface (PDB 4KBB) with seven electrostatic bonds. The recognition mechanism is mostly based on strong protein-protein hydrophobic interactions. The small difference in contact surface area and number of electrostatic interactions may be explained by the sequence variation between hSyt1 and rSyt2, in particular towards the C-terminal half of the peptide.

The third receptor contained in the H_{C}/TAB-3R structure corresponds to the GD1a carbohydrate, for which clear electron density was observed from Gal2 to Sia5 (FIG. 4). No electron density was visible for Glc1 and Sia6, as may be expected from non-interacting flexible carbohydrate moieties. The ganglioside-receptor binding site has been studied in details, and the crystal structure of H_{C}/B in complex with GD1a had confirmed the preference of this serotype for the terminal Sia(α2-3)Gal moiety (Bertnsson et al., 2013; Rummel, 2016). TriRecABTox was designed to integrate the H_{C}/B binding pocket, and comparison of the two structures (FIG. 7) shows that key residues of the binding pocket (S1260, W1262, Y1263) are fully conserved and interact with GD1a as per the native toxin. Most of the binding site remains unchanged when compared to the GD1a-bound H_{C}/B, with few noticeable exceptions. In H_{C}/TAB-3R, the side chain of N1122 faces away from the ligand while its H_{C}/B equivalent, N1105, makes a direct hydrogen bond with Sia5. This is somewhat compensated by the position of 11257 that shows stronger hydrophobic interaction (at a distance of 4.3 Å) with Sia5 in H_{C}/TAB-3R compared to I1240 in the H_{C}/B:GD1a structure (where they are 7 Å apart).

Overall the results obtained from the H_{C}/TAB-3R crystal structure confirms that a single TriRecABTox molecule is able to simultaneously bind to SV2 receptor, synaptotagmin receptor and its ganglioside receptors in a manner that replicates the binding mechanisms of the parent BoNT/A and /B.

### Production and Characterisation of the Full-Length, Inactive TriRecABTox.

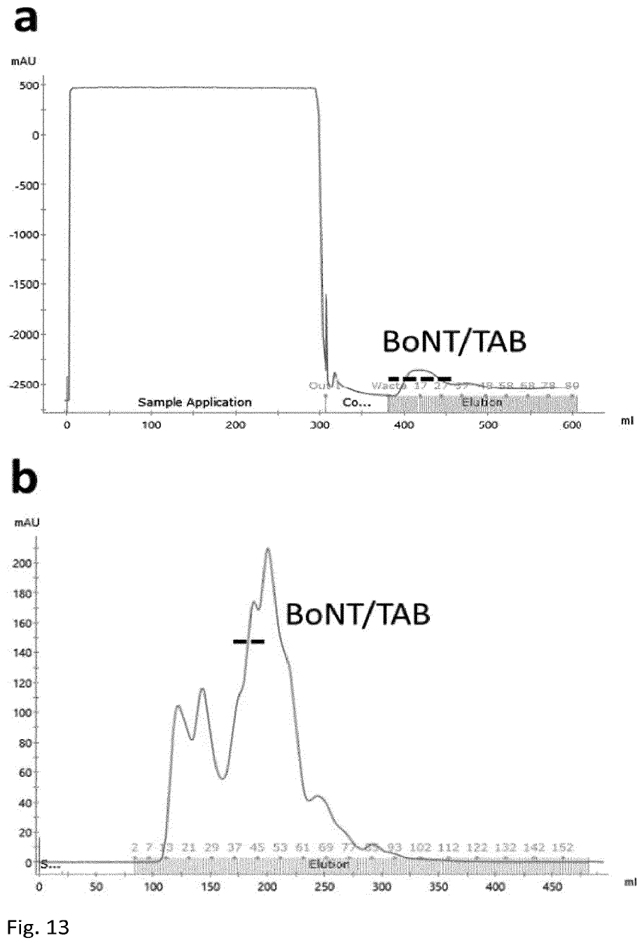

Having established the binding capability of H_{C}/TAB the inventors went on to express and purify the full-length, catalytically inactive, TriRecABTox (BoNT/TAB; SEQ. ID. No. 3). For this purpose, the inventors designed a synthetic gene encoding for 1311 amino acids and containing the three BoNT functional domains, with LC and H_{N} corresponding to the BoNT/A domains, associated with H_{C}/TAB. Three mutations at the catalytic site were included for safety considerations (E2240/R363A/Y366F) (Rossetto et al, 2001; Binz et al, 2002). As per the H_{C}/TAB construct described above, the protein sequence was codon-optimised for expression in E. coli, and cloned into pET-28a(+) with a N-terminal poly-histidine tag. Details are provided in the methods section. The inventors showed that BoNT/TAB could be expressed as a soluble protein of approximately 152 kDa. The initial method used for purification yielded limited amount of non-homogenous material (FIG. 8; FIG. 13), but further purification using methods such as ion exchange or hydrophobic interaction chromatography should help obtain purer material, and eliminate the residual host cell proteins visible by gel electrophoresis. Such method was used recently to produce a recombinant BoNT/B construct with more than 80% purity (Elliot et al., 2017).

Additional characterisation was carried out and confirmed the presence of the histidine-tag, and although the reaction with the probing antibody was very weak compared to the controls (FIG. 8B), a faint band was discernable at the right size. The assay also showed cross-reaction with a contaminant of approximately 70 kDa. Furthermore, BoNT/TAB reacted conclusively with in-house anti-sera raised against H_{C}/A (FIG. 8C) and H_{C}/B (FIG. 8C), as was expected, since it should contain epitopes from both binding domains.

### Controlled Activation of TriRecABTox.

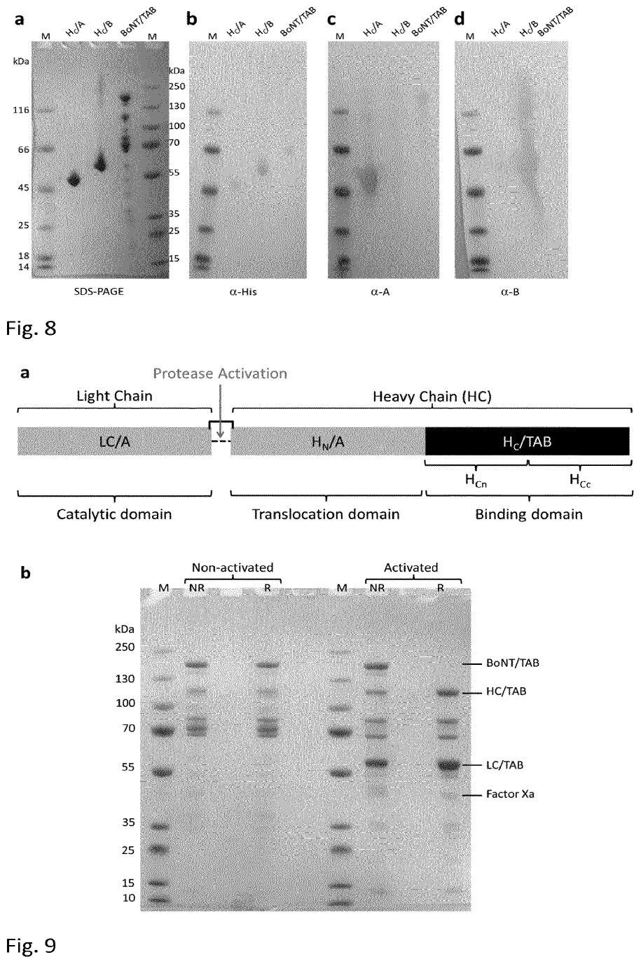

BoNT/TAB was designed with a Factor Xa cleavage site, IEGR [442-445], between the light and heavy chains (FIG. 9A) since activation into a di-chain form is necessary to obtain a fully active toxin. The full-length BoNT/TAB sample (SEQ. ID. No. 5) described above was used to carry out an activation assay. Despite the sample's heterogeneity, full activation was achieved after incubation of BoNT/TAB with Factor Xa, at a ratio of 1 μg protease to 50 μg of toxin, overnight at 4° C. (FIG. 9B). Gel electrophoresis showed separation of BoNT/TAB into two fragments of approximately 100 and 50 kDa when run in presence of a reducing agent, most likely corresponding to HC and LC, respectively. These two chains are held together by a disulphide bridge between C430 and C458, explaining the single band at approximately 150 kDa in non-reducing condition. Bands corresponding to HC and LC were also visible in the non-reducing sample and may have been caused by some level of reduction of the disulphide bridge during sample preparation, however these bands were clearly not visible in the non-activated control.

Altogether the activation assay first provided evidence that the protein produced corresponds to the engineered BoNT/TAB, and secondly that the activation step into a di-chain molecule could be successfully managed. Therefore such step may be included in the production of active full-length TriRecABTox.

### Optimisation of BoNT/TAB

### Material and Methods

Constructs. The cDNA encoding H_{C}/TAB and variants were cloned by GenScript (NJ, USA), in a pET28(a) vector as described previously. BoNT/TAB2.1.3 was cloned in a pET29(a) vector by Toxogen GmbH (Hannover, Germany).

Protein expression and purification. As described previously for H_{C}/TAB variants. BoNT/TAB2.1.3 was produced by Toxogen GmbH (Hannover, Germany), with a protocol similar to the one used for H_{C}/TAB (affinity chromatography and gel filtration). In addition, activation and tag removal of BoNT/TAB2.1.3 was performed with Thrombin at a concentration of 0.05 U/μg, and BoNT/TAB2.1.3 was further purified by gel filtration. Samples were stored in 25 mM HEPES pH 7.2 with 200 mM NaCl, and 5% glycerol.

Protein characterisation. As described previously (gel electrophoresis using NuPAGE 4-12% Bis-Tris gels).

X-ray crystallography. Samples for crystallisation were prepared by pre-incubation for 15 min at room temperature of H_{C}/TAB2.1 at 6.5 mg/ml, with SV2C-L4 at 1 mg/ml (recombinant human SV2C extracellular loop-4 [residues 475-565], 1 mM hSytI peptide (GEGKEDAFSKLKEKFMNELHK (SEQ ID NO: 14), synthesised by GenScript, USA) and 4 mM GD1a oligosaccharide (Elicityl, France). Crystals were grown with 200 nl of sample mixed with 100 nl of reservoir solution consisting of 20% v/v polyethylene glycol 3350, 0.2 M Potassium citrate (JCSG-plus screen B12, Molecular Dimensions, United Kingdom) using a sitting drop set-up and incubated at 21° C. Crystals appeared within 1 week and were transferred to a cryo-loop and frozen in liquid nitrogen. Diffraction data were collected at station 104 of the Diamond Light Source (Didcot, UK), equipped with a PILATUS-6M detector (Dectris, Switzerland). A complete dataset to 1.4 Å was collected from a single crystal at 100° K. Raw data images were processed and scaled with DIALS (Gildea et al, 2014), and AIMLESS (Evans, 2006) using the CCP4 suite 7.0 (CCP4, 1994).

Molecular replacement was performed with the structure of H_{C}/TAB determined previously in PHASER (McCoy et al., 2007). The working models were refined using REFMAC5 (Murshudov et al, 2011) and manually adjusted with COOT (Emsley et al., 2010). Water molecules were added at positions where Fo-Fc electron density peaks exceeded 3σ, and potential hydrogen bonds could be made. Validation was performed with MOLPROBITY (Chen et al., 2010). Ramachandran statistics show that 97.0% of all residues are in the most favoured region, with a single outlier in the disallowed region. Crystallographic data statistics are summarized in Table X1.

Production of an Optimised H_{C}/TAB, H_{C}/TAB2.1

The crystal structure of H_{C}/TAB bound to its three receptors was analysed in order to identify potential sites that could be modified to improve the molecule's stability and function.

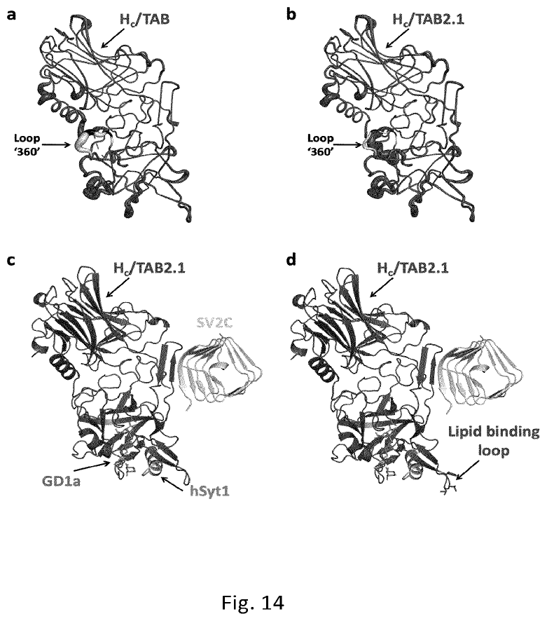

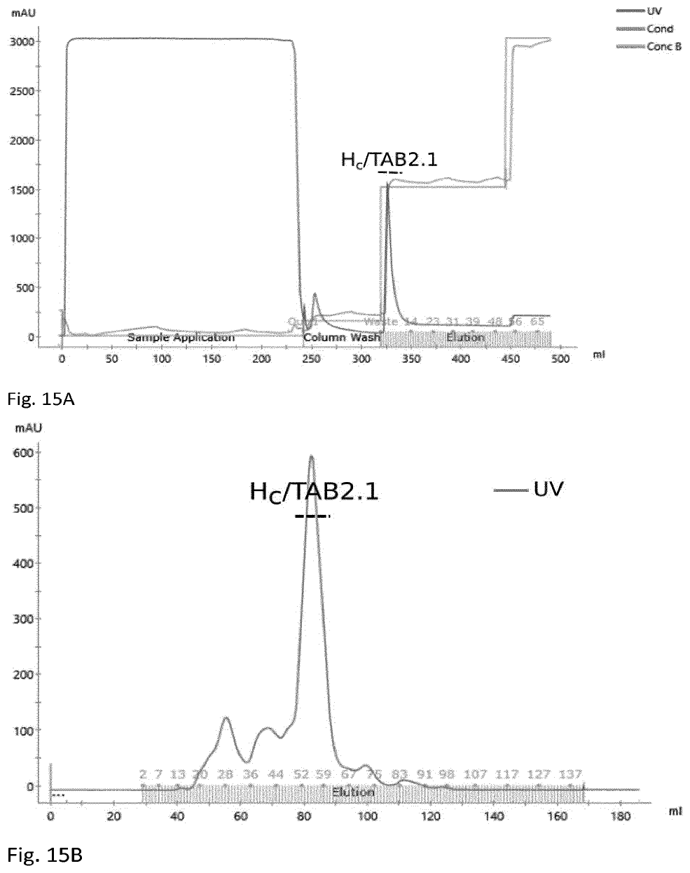

In particular, analysis of the local temperature factors (B-factor) within a crystal structure may be interpreted as an indication of the local stability of a protein, with high B-factor suggestive of a disorderly region. From this analysis, a loop at the interface between the two subdomains of H_{C}/TAB, labelled ‘loop 360’, consisting of residues D357 to N362 (SEQ ID: No. 6), was considered for optimisation (See FIG. 14). Residues G360 and N362 (SEQ ID. No.1) were modified to their equivalent residues in BoNT/B and mutated to P360 and Y362 respectively, to be incorporated in the sequence of a new construct labelled H_{C}/TAB2.1 (SEQ. ID. No. 6).

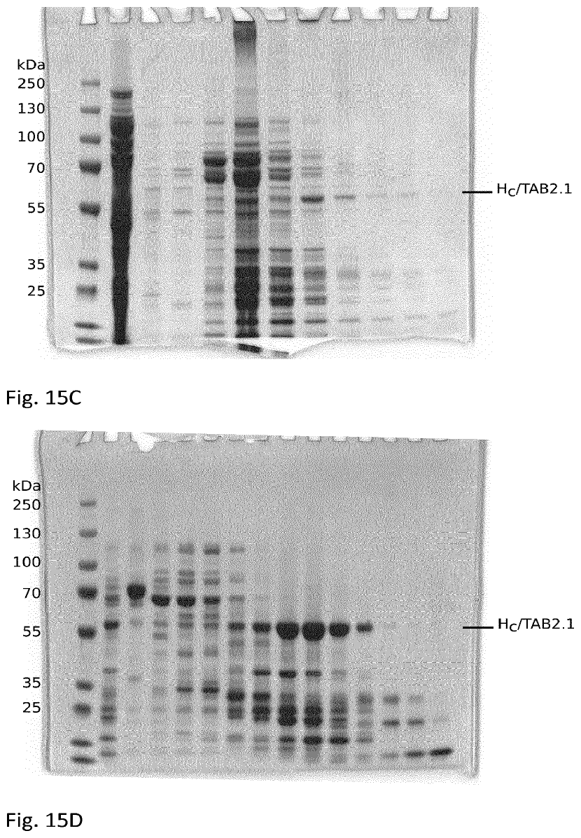

The plasmid for this new construct was prepared by site-directed mutagenesis (GenScript, USA) and used for recombinant expression of H_{C}/TAB2.1 in E. coli. The protocol used was the same as for the production of H_{C}/TAB (see original method section for expression and purification). We showed that H_{C}/TAB2.1 could be expressed and partially purified using affinity chromatography and size exclusion techniques (FIG. 15). The sample presented some low molecular weight contaminants that likely correspond to residual host cell proteins. Additional purification steps using methods such as ion exchange or hydrophobic interaction chromatography should help obtain a sample of higher purity.

The purified H_{C}/TAB2.1 (SEQ. ID. No. 6) was used in co-crystallisation trials with the human SV2C luminal domain [residues 475-565], the human Syt1 peptide [residues 34-53] and the GD1a carbohydrate. Crystals were obtained that diffracted to high resolution (1.4 Å) and a complete dataset could be collected (Table 2). The structure was solved by molecular replacement using the crystal structure of H_{C}/TAB bound to it three receptors (H_{C}/TAB-3R). The new structure presented all the elements already visible in H_{C}/TAB-3R and provided experimental evidence that H_{C}/TAB2.1 can bind to the three receptors simultaneously, as per H_{C}/TAB. Analysis of the B-factor, showed an improved stability for loop ‘360’ (D357 to Y362; FIG. 14). Overall, the behaviour of H_{C}/TAB2.1 was similar to that of HC/TAB, with both constructs showing comparable profiles in terms of yield and purity.

TABLE X1
X-ray crystallography: data collection and refinement statistics

| | H_{C}/TAB2.1-SV2C-hSytl-GD1a complex |
|---|---|
| Data collection | |
| Space group | P2₁2₁2₁ |
| Cell dimensions | |
| a, b, c (Å) | 43.8, 117.5, 141.5 |
| α, β, γ (°) | 90.0, 90.0, 90.0 |
| Resolution (Å) | 1.4-60.6 (1.40-1.56)* |
| No. total/unique reflections | 1,055,932/79,953 |
| R_{merge} | 0.084 (1.070)* |
| Rₚᵢₘ | 0.024 (0.364)* |
| CC_{1/2} | 0.99 (0.716)* |
| I/σl | 16.0 (1.8)* |
| Completeness (%) | 95.1 (70.1)* |
| Redundancy | 13.2 (9.3)* |
| Refinement | |
| R_{work}/R_{free} (%) | 17.7/20.4 |
| R.m.s. deviations | |
| Bond lengths (Å) | 0.118 |
| Bond angles (°) | 1.60 |

*Values in parentheses are for highest-resolution shell.

### Production of a More Soluble Variant, H_{C}/TAB2.1.3

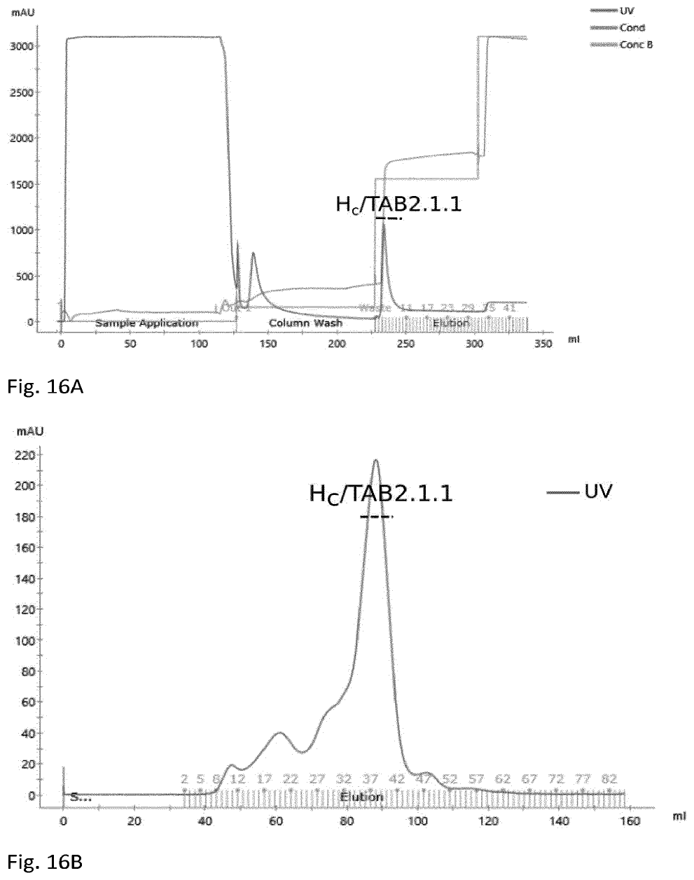

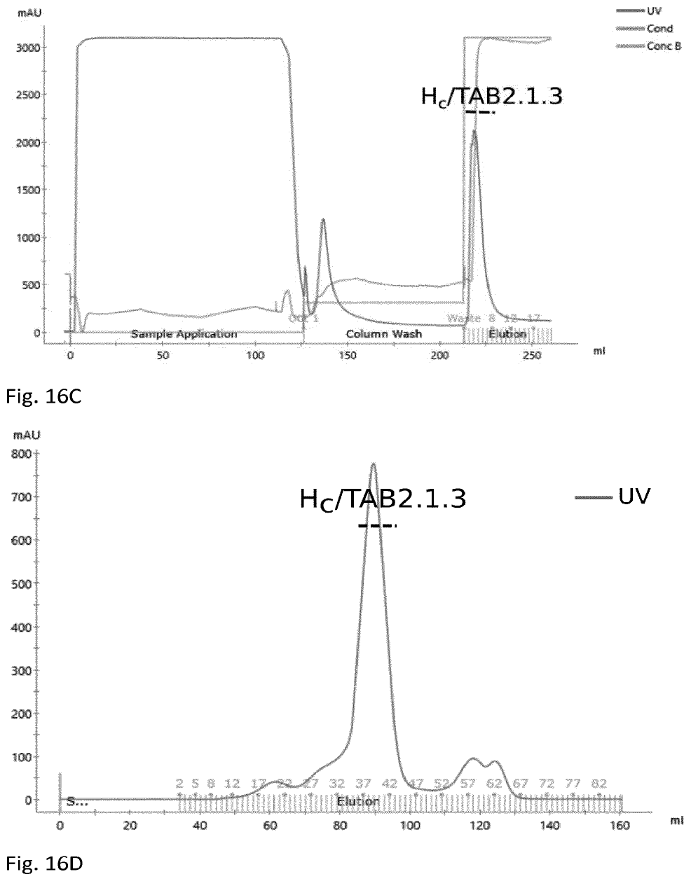

In order to prepare for future functional analysis of the H_{C}/TAB variants, H_{C}/TAB2.1 was adapted to be compatible with a sortase ligation experiment described recently (Zhang et al, 2017). This experiment allows for a safe and controlled reconstruction of a full-length active BoNT that can be used to test activity. This construct corresponds to a N-terminal truncated H_{C}/TAB2.1 with a cleavable N-terminal His-tagged, and was labelled H_{C}/TAB2.1.1 (SEQ. ID. No. 8). The clone for H_{C}/TAB2.1.1 was prepared (GenScript), used for expression and purification as described previously (FIG. 16). We showed that H_{C}/TAB2.1.1 could be expressed and partially purified using affinity chromatography and size exclusion techniques. The sample presented some low molecular weight contaminants that likely correspond to residual host cell proteins. Additional purification steps using methods such as ion exchange or hydrophobic interaction chromatography should help obtain a sample of higher purity.

Further analysis of the structural features of H_{C}/TAB2.1 highlighted the presence of a surface-exposed hydrophobic loop which protrudes from the rest of the protein (residues 389-393, SEQ ID: No. 6; FIG. 14d). In addition, this loop was recently identified as a lipid-binding element in BoNT/B and other serotypes (Stern et al, 2018). We hypothesised that this hydrophobic region could hinder the solubility of H_{C}/TAB, thus a new construct was designed in which this loop was truncated and replaced with a dual-asparagine motif to enhance solubility. This construct was labelled H_{C}/TAB2.1.3 (SEQ.ID. No. 10). The clone for H_{C}/TAB2.1.3 was prepared (GenScript), used for expression and purification as described previously (FIG. 16). We showed that H_{C}/TAB2.1.3 could be expressed and partially purified using affinity chromatography and size exclusion techniques. The sample presented some low molecular weight contaminants that likely correspond to residual host cell proteins. Additional purification steps using methods such as ion exchange or hydrophobic interaction chromatography should help obtain a sample of higher purity. Noticeably, H_{C}/TAB2.1.3 showed better expression yield and solubility compared to H_{C}/TAB2.1.1 (FIG. 16).

### Production of a Full-Length, Active BoNT/TAB2.1.3

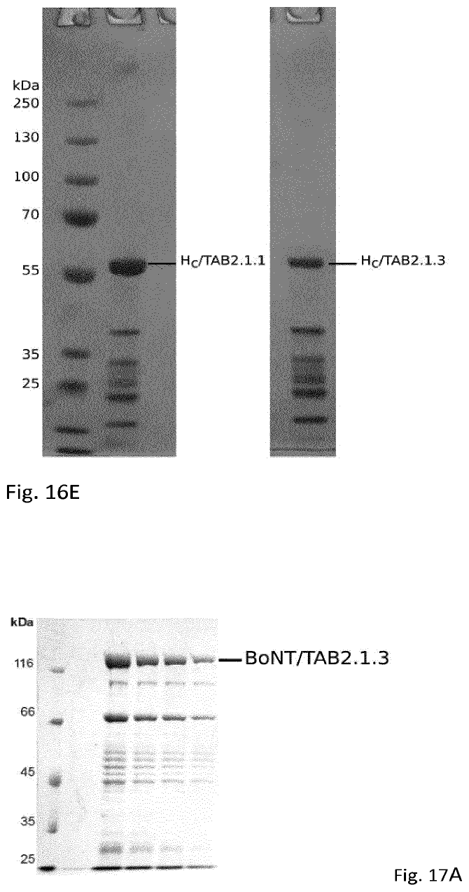

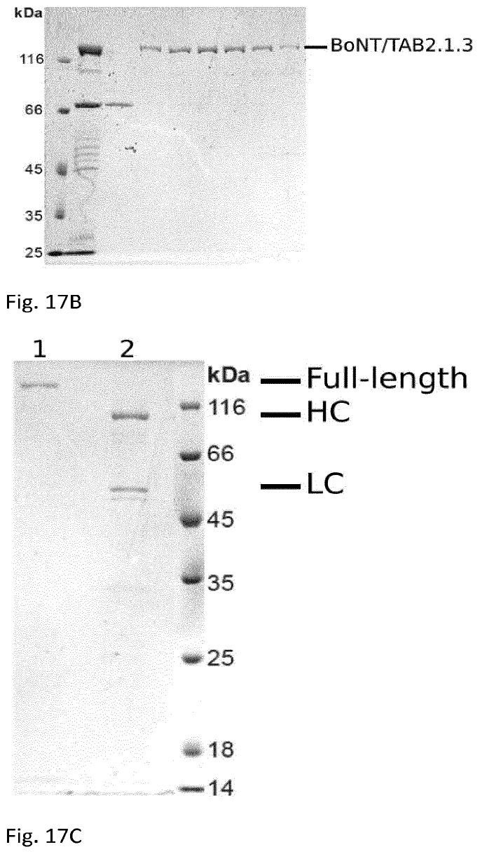

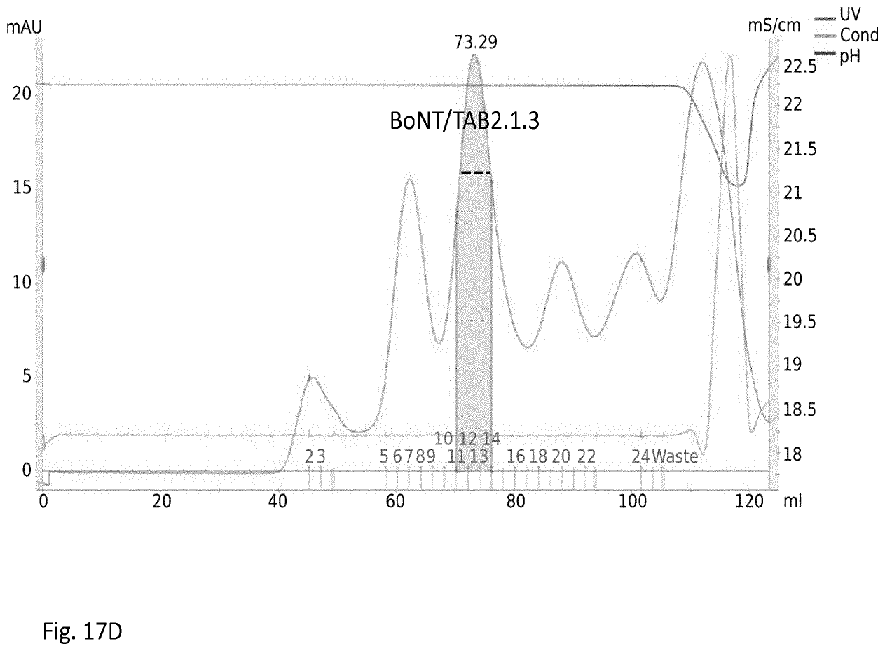

In order to prepare for future functional analysis of BoNT/TAB, a full-length active variant based on the H_{C}/TAB2.1.3 construct was produced and labelled BoNT/TAB2.1.3 (SEQ. ID. No. 12). All steps of the production were carried out in a licensed facility, under contract agreement, at Toxogen GmbH (Hannover, Germany). BoNT/TAB2.1.3 was cloned in a pET29(a) vector and included cleavable C-terminal Strep-and poly-histidine tags, as well as an engineered thrombin cleavage site between the HC and LC domains (SEQ. ID. No. 13), for activation of the product, as described previously. BoNT/TAB2.1.3 could be expressed as a soluble protein, purified and activated with thrombin (FIG. 17). The method used for purification included affinity chromatography and gel filtration, and led to a BoNT/TAB2.1.3 product with >90% purity.

### FUTURE EXPERIMENTS

### Receptor Binding Assays

Assays will be performed where the receptor-binding properties of BoNT/TAB will be compared to BoNT/A and/or BoNT/B.

For example, ganglioside receptor-binding assays will be carried out that are adapted from previously described methods. Briefly, in this ELISA the ganglioside receptor of interest (GT1b, GD1b, GD1a, or GM1a) is immobilised on a 96-well microplate (Chen et al., 2008; Willjes et al., 2013), the toxins (or their binding domain) are then applied, and the bound material probed with a monoclonal anti poly-Histidine antibody conjugated to horse radish peroxidase (HRP). This qualitative approach should provide enough information to confirm that the ganglioside receptor-binding characteristics of BoNT/TAB are similar to that of BoNT/B.

Ganglioside receptor binding ELISA. Gangliosides GT1b, GD1b, GD1a, and GM1a are purchased from Carbosynth (Compton, UK). Gangliosides are diluted in methanol to reach a final concentration of 2.5 μg/ml; 100 μL (0.25 μg) is applied to each well of a 96-well PVC assay plates. After evaporation of the solvent at 21° C. (overnight), the wells are washed (3×) with 200 μL of PBS/0.1% (w/v) BSA. Nonspecific binding sites are blocked by incubation for 2 h at 21° C. in 200 μL of PBS/2% (w/v) BSA. Binding assays are performed in 100 μL of PBS/0.1% (w/v) BSA per well for 2 h at 4° C. containing the samples (serial 3-fold dilution ranging from 6 μM to 0.003 μM). Following incubation, wells are washed 3× with PBS/0.1% (w/v) BSA and then incubated with an HRP-anti-His antibody (ThermoFisher #MAI-80218) at a 1:2000 dilution (100μ1/well) for 1 hat 4° C. Finally, after three washing steps with PBS/0.1% (w/v) BSA, bound samples are detected using Ultra TMB (100 μL/well). The reaction is terminated after incubation for 5 min at 21° C. by addition of 100 μL of 1M sulphuric acid. Absorbance at 450 nm is measured with a Tecan Infinite 200 (Mannedorf, Switzerland). Results are analysed with Prism (GraphPad, La Jolla, CA, USA), using a non-linear binding fit.

In order to assess the binding properties to the synaptotagmin receptor, isothermal titration calorimetry (ITC) will be performed, similarly to the assay described by Berntsson et al. (2013). Binding of the hSyt peptides to the toxins will be measured and should provide affinity values (K_{d}) confirming that BoNT/TAB can bind to the receptor, analogously to BoNT/B.

Isothermal titration calorimetry. Samples are prepared by an additional size exclusion chromatography step (Superdex200, GE Healthcare, Sweden) in 20 mM potassium phosphate pH 7.0, 0.15 M NaCl. Association of Syt peptides to BoNT or their binding domains is measured on an ITC200 (GE Healthcare, Sweden) at 25° C. and 750 rpm. A 200 μL solution of protein (at 20 μM) is added to the cell. Binding is measured upon the addition of peptide (GenScript, USA) with 16 stepwise injections of 2.5 μL each, at a concentration of 200 μM. The first titration is set to 0.5 μL and is subsequently deleted in the data analysis. Data is analysed with the Origin software provided by the manufacturer

The binding to SV2C will be assessed using a pull-down assay such as the one described by Benoit et al. (2014). Briefly, the tagged toxin and non-tagged receptor (or inversely) will be incubated together and loaded onto a Ni-sepharose, then washed and eluted. Results will be visualised by SDS-PAGE.

### Digit Abduction Score (DAS) Assay

The potency of BoNT preparation can be evaluated using a mouse Digit Abduction Score (DAS) assay (Broide et al., 2013). This assay measures in vivo the local muscle-weakening efficacy of the toxin after intramuscular injection into mouse or rat hind limb skeletal muscle. The toxin elicits a measurable dose-dependent decrease in the animal's ability to produce a characteristic hind limb startle response. This non-lethal method has been used regularly to estimate the pharmacological properties of different BoNT serotypes or derivatives, such as the recently described recombinant BoNT/B molecules (Elliot et al., 2017). A similar methodology will be used to assess the potency and duration of effect of BoNT/TAB, compared to BoNT/A or /B.

### Discussion

In this study the inventors described how the structural and molecular details of the binding mechanism of BoNT/A and /B were used to engineer a new molecule, TriRecABTox, that possesses enhanced cell recognition capability. A rigorous multi-dimension comparison of BoNT/A and /B structures allowed the inventors to identify the key elements necessary to keep an intact toxin scaffold on which to integrate the receptor binding sites for SV2, synaptotagmin and a ganglioside, in a single molecule. The newly created design, consisting of an alternation of BoNT/A and /B elements, was optimised by including adaptive mutations or deletions to compensate for the newly created non-natural intramolecular interfaces. Such modifications were deemed necessary to ensure that the engineered toxin, BoNT/TAB, could be produced as a soluble protein with the correct structure and required activity.

The inventors first assessed the stability of the design by producing the binding domain on its own, H_{C}/TAB, which holds the modified receptor recognition function, via recombinant expression in E. coli. H_{C}/TAB was expressed with a N-terminal poly-histidine tag as a soluble protein that could be partially purified, thus demonstrating the viability of the engineered construct. In a second step, the inventors proceeded with the production of the full-length BoNT/TAB construct, in a catalytically inactive form. Again, the inventors showed that it could be expressed as a soluble protein of 153 kDa and partially purified with standard liquid chromatography techniques. Presence of the poly-histidine tag on both H_{C}/TAB and BoNT/TAB allowed their purification by affinity chromatography with a Ni-sepharose matrix. Other affinity methods may be used and include an affinity tag that should be preferentially positioned on the N-terminal end of the protein in order to prevent interference with receptor binding. Although the initial preparation showed heterogeneous sample purity, optimisation of the purification process should lead to a product of pharmaceutical standards. It should be added that the active form of BoNT/TAB would have a similar overall structure and binding properties to the inactive molecule used in the present study. The inventors contracted Toxogen GmbH (Hannover, Germany) to produce an active version of BoNT/TAB (BoNT/TAB2.1.3) that was purified successfully with a removable C-terminal tag, so as to not interfere with receptor binding.

In addition, post-translational cleavage of the single-chain BoNT into a di-chain molecule is an essential step for the toxin's activity (DasGupta and Sathyamoorthy, 1985; Shone et al, 1985). While the native toxin is usually activated by a host protease, any recombinant BoNT product needs to be processed with an exopeptidase. Early work on the toxin showed that trypsin could non-specifically cleave BoNT/A to an active di-chain form (Shone et al., 1985), however this may result in unwanted additional degradation of the toxin. More recently, recombinant technologies have allowed the engineering of specific protease recognition motifs within a protein of interest, thus providing better control on the activation strategy of BoNT (Sutton et al, 2005). Here the inventors included a Factor Xa site between LC and HC and observed complete activation of the toxin, thus demonstrating the effectiveness of this enzyme. Future production of BoNT/TAB should incorporate a purification stage that allows for activation of the toxin, followed by removal of the exoprotease from the final product. While Factor Xa appears adequate, other enzymes may be tested and prove successful in achieving acceptable yield of activation. The inventors contracted Toxogen GmbH (Hannover, Germany) to produce an active version of BoNT/TAB (BoNT/TAB2.1.3) that was successfully activated with the thrombin exoprotease and purified to homogeneity.

As a mean to verify the structural integrity of H_{C}/TAB and confirm its enhanced functionality, the inventors co-crystallised the purified sample in complex with human SV2C, human Syt1 and the GD1a carbohydrate. The X-ray crystal structure of the complex was solved to high resolution (1.5 Å), and provided conclusive experimental evidence that a single molecule of H_{C}/TAB could bind to all three receptors simultaneously. Furthermore, comparison to the known structures of H_{C}/A and H_{C}/B with their respective receptors showed that H_{C}/TAB follows an almost identical mechanism of binding.

While the crystal structure demonstrated that H_{C}/TAB could fulfil its purpose, at least in vitro, additional biochemical experiments need to be performed to fully characterise its receptor binding properties. These will include pull-down and ITC assays with the protein receptors, and ganglioside receptor binding ELISA. BoNT/TAB is expected to perform similarly to BoNT/A for SV2 receptor binding, and similarly to BoNT/B with regards to ganglioside receptor and synaptotagmin receptor binding. Additionally, in vivo experiments will provide the main indications on the true potential of BoNT/TAB as a therapeutic. The mouse DAS assay has classically been used to assess BoNT preparations (Broide et al., 2013) and should allow the inventors to determine the efficacy and duration of action of our molecule compared to the currently available products.

Additionally, the design of BoNT/TAB may be further optimised by modifying some sequence elements to improve its biochemical properties and stability. Such alterations may include deletions or mutations that lead to a soluble BoNT still able to simultaneously bind to three receptors. The inventors successfully produced a more stable variant (H_{C}/TAB2.1) and a more soluble variant with higher production yield (H_{C}/TAB2.1.3).

It should be added that from a safety perspective, BoNT/TAB do not represent a novel threat since it is derived from two existing serotypes. It is expected to be recognised by currently available anti-toxins, such as the Botulism Antitoxin Heptavalent BAT or other approved antidotes for BoNT/A and /B.

Serotypes A and B are the only approved BoNTs available on the market. While BoNT/A is the main toxin used therapeutically, molecules with lower immunogenicity and high efficacy would provide safer alternatives (Naumann et al., 2013). Multiple attempts have been made at improving the properties of BoNTs in order to increase their pharmacological potential (Masuyer et al., 2014). A recent successful example include the study by Tao et al. (2017) where mutations engineered in key positions of BoNT/B (E1191M/S1199Y) gave the toxin higher affinity for the human synaptotagmin2 receptor, and showed approximately 11-fold higher efficacy in blocking neurotransmission compared to the wild type. Another approach to improve BoNT efficacy was taken by Elliott et al. (2017) where they analysed the effect of a single mutation (S201P) known to increase the catalytic activity of BoNT/B on its substrate. In this case, the BoNT/B mutant did not present any advantage over the wild type in multiple cell-based assays and in vivo. Altogether these two studies on BoNT/B suggest that the limiting step in the toxin's efficacy resides in the initial neuronal recognition rather than the later intracellular activity.

Earlier studies intending to combine the binding properties of one serotype with the catalytic activity of another led to the design of chimeric molecules where whole domains were swapped (Wang et al., 2008, 2012; Rummel et al., 2011). More particularly, Rummel et al. (2011) and Wang et al. (2012) designed and tested analogous molecules consisting of the H_{C}/B domain associated with the H_{N}+LC domains of BoNT/A. These recombinant toxins were reported to display enhanced potency and induced a lengthier effect in mice compared to the wild type BoNT/A (Kutschenko et al., 2017). Similar observations were obtained when assessing a construct consisting of the C-terminal subdomain (H_{Cc}) of BoNT/B coupled with the complementary domains of serotype A (i.e. LC+H_{N}+H_{Cn}), and which showed a four-fold higher potency compared to the wild-type (Rummel et al., 2011). All the molecules described above had in common the fact that they would only recognise the two receptors of BoNT/B, synaptotagmin and ganglioside. These results suggest that prolonged effect and higher efficacy may be obtained thanks to a greater intake of LC/A permitted by the higher prevalence of the BoNT/B receptors on neurons. In addition, these chimeric molecules did not take into account the possible inter-domain intra-molecular clashes that may arise from combining domains from different serotypes, and which may affect the potential of these products.

Taking into considerations the results from the latest studies on BoNT engineering, it appears clear that modifying initial cellular recognition is one of the most efficient ways to enhance the pharmacological properties of the therapeutic product. Therefore BoNT/TAB, a single product successfully engineered to recognise SV2 receptor together with the BoNT/B receptors, synaptotagmin and ganglioside, represents a great potential and could yet be more efficacious than the wild type BoNT/A and /B.

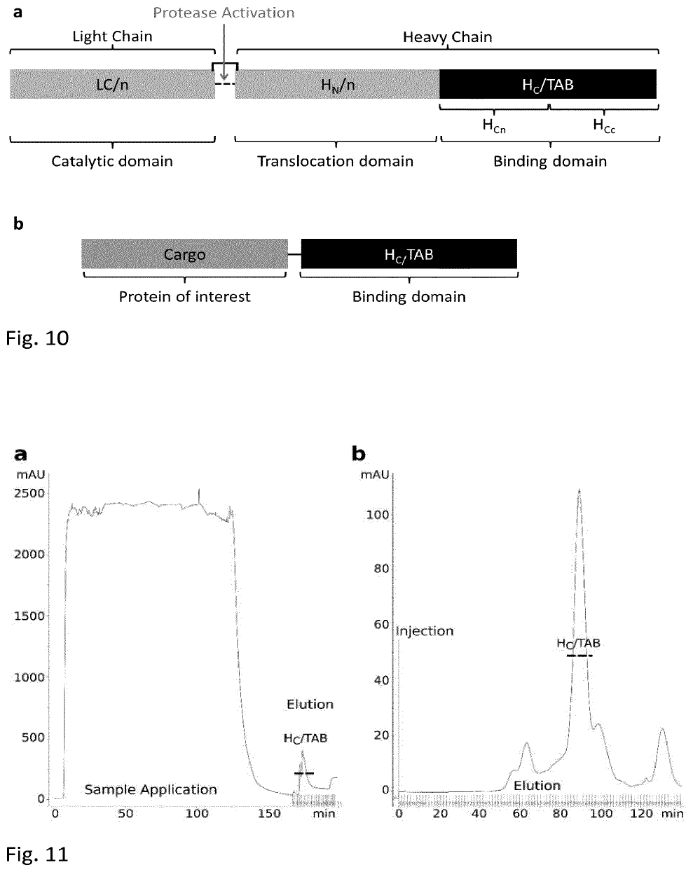

The main innovation in BoNT/TAB is the design of the binding domain allowing multiple receptor interactions. Current evidence hints that association of H_{C}/TAB with the translocation and catalytic domains of BoNT/A should provide the molecule with the strongest potency (as designed in BoNT/TAB). However, H_{C}/TAB may still be of interest when combined with the functional domains of other serotypes (FIG. 10a). In addition, H_{C}/TAB may also be coupled with other proteins of interest (FIG. 10b) to be used as a pharmacological tool to investigate synaptic processes. The in vivo assays to be performed with BoNT/TAB should clarify its utility for such purpose.

### REFERENCES

Benoit RM, Frey D, Hilbert M, Kevenaar JT, Wieser MM, Stirnimann CU, McMillan D, Ceska T, Lebon F, Jaussi R, Steinmetz MO, Schertler GF, Hoogenraad CC, Capitani G, Kammerer RA. 2014. Structural basis for recognition of synaptic vesicle protein 2C by botulinum neurotoxin A. Nature. 505:108-111.
Bentivoglio AR, Del Grande A, Petracca M, Ialongo T, Ricciardi L. 2015. Clinical differences between botulinum neurotoxin type A and B. Toxicon. 107:77-84.
Berntsson RP, Peng L, Dong M, Stenmark P. 2013. Structure of dual receptor binding to botulinum neurotoxin B. Nat Commun. 4:2058.
Binz T, Bade S, Rummel A, Kollewe A, Alves J. 2002. Arg(362) and Tyr(365) of the botulinum neurotoxin type a light chain are involved in transition state stabilization. Biochemistry 41:1717-1723.
Binz T, Rummel A. 2009. Cell entry strategy of clostridial neurotoxins. J Neurochem. 109:1584-1595.
Broide RS, Rubino J, Nicholson GS, Ardila MC, Brown MS, Aoki KR, Francis J. 2013. The rat Digit Abduction Score (DAS) assay: a physiological model for assessing botulinum neurotoxin-induced skeletal muscle paralysis. Toxicon. 71:18-24.
Chai Q, Arndt JW, Dong M, Tepp WH, Johnson EA, Chapman ER, Stevens RC. 2006. Structural basis of cell surface receptor recognition by botulinum neurotoxin B. Nature. 444:1096-1100.
Chen C, Baldwin MR, Barbieri JT. 2008. Molecular basis for tetanus toxin coreceptor interactions. Biochemistry. 47:7179-7186.
Chen S. 2012. Clinical uses of botulinum neurotoxins: current indications, limitations and future developments. Toxins (Basel). 4:913-39.
Chen VB, Arendall WB, Headd JJ, Keedy DA, Immormino RM, Kapral GJ, Murray LW, Richardson JS, Richardson DC. 2010. MolProbity: all-atom structure validation for macromolecular crystallography. Acta Crystallogr D66:12-21.
Collaborative Computational Project, Number 4. 1994. The CCP4 suite: programs for protein crystallography. Acta Crystallogr D50:760-763.
DasGupta BR, Sathyamoorthy VS. 1985. Separation, purification, partial characterization and comparison of the heavy and light chains of botulinum neurotoxin types A, B, and E. J Biol Chem 260:10461-10466.
Dong M, Richards DA, Goodnough MC, Tepp WH, Johnson EA, Chapman ER. 2003. Synaptotagmins I and II mediate entry of botulinum neurotoxin B into cells. J. Cell Biol. 162:1293-303.
Dong M, Yeh F, Tepp WH, Dean C, Johnson EA, Janz R, Chapman ER. 2006. SV2 is the protein receptor for botulinum neurotoxin A. Science. 312:592-596.
Dressler D, Bigalke H. 2005. Botulinum toxin type B de nova therapy of cervical dystonia: frequency of antibody induced therapy failure. J Neural. 252:904-907.
Elliott M, Maignel J, Liu SM, Favre-Guilmard C, Mir I, Farrow P, Hornby F, Marlin S, Palan S, Beard M, Krupp J. 2017. Augmentation of VAMP-catalytic activity of botulinum neurotoxin serotype B does not result in increased potency in physiological systems. PLoS One. 12:e0185628.
Emsley P, Lohkamp B, Scott WG, Cowtan K. 2010. Features and development of Coot. Acta Crystallogr D66:486-501.
Evans P. 2006. Scaling and assessment of data quality. Acta Crystallogr D62:72-82.
Gildea RJ, Waterman DG, Parkhurst JM, Axford D, Sutton G, Stuart DI, Sauter NK, Evans G, Winter G. 2014. New methods for indexing multi-lattice diffraction data. Acta Crystallogr. D70:2652-2666.
Hamark C, Berntsson RP, Masuyer G, Henriksson LM, Gustafsson R, Stenmark P, Widmalm G. 2017. Glycans Confer Specificity to the Recognition of Ganglioside Receptors by Botulinum Neurotoxin A. J Am Chem Soc. 139:218-230.
Hatheway CL. 1990. Toxigenic clostridia. Clin. Microbial. Rev. 3:66-98.
Jin R, Rummel A, Binz T, Brunger AT. 2006. Botulinum neurotoxin B recognizes its protein receptor with high affinity and specificity. Nature. 444:1092-1095.
Krissinel E. 2015. Stock-based detection of protein oligomeric states in jsPISA, Nucl. Acids Res. 43:W314-9.
Kutschenko A, Reinert MC, Krez N, Liebetanz D, Rummel A. 2017. BoNT/AB hybrid maintains similar duration of paresis as BoNT/A wild-type in murine running wheel assay. Neurotoxicology. 59:1-8.
Lacy DB, Stevens RC. 1999. Sequence homology and structural analysis of the clostridial neurotoxins. J Mai Biol. 291:1091-104.
Lange 0, Bigalke H, Dengler R, Wegner F, deGroot M, Wohlfarth K. 2009. Neutralizing antibodies and secondary therapy failure after treatment with botulinum toxin type A: much ado about nothing? Clin. Neuropharmacol. 32:213-218.
Masuyer G, Chaddock JA, Foster KA, Acharya KR. 2014. Engineered botulinum neurotoxins as new therapeutics. Annu Rev Pharmacol Toxicol. 54:27-51.
Mahrhold S, Rummel A, Bigalke H, Davletov B, Binz T. 2006. The synaptic vesicle protein 2C mediates the uptake of botulinum neurotoxin A into phrenic nerves. FEBS Lett. 580:2011-2014.
McCoy AJ, Grosse-Kunstleve RW, Adams PD, Winn MD, Storoni LC, Read RJ. 2007. Phaser crystallographic software. J Appl Crystallogr 40:658-674.
Montal M. 2010. Botulinum neurotoxin: a marvel of protein design. Annu Rev Biochem. 79:591-617.
Murshudov GN, Skubak P, Lebedev AA, Pannu NS, Steiner RA, Nicholls RA, Winn MD, Long F, Vagin AA. 2011. Refmac5 for the refinement of macromolecular crystal structures. Acta Crystallogr D67:355-367.
Naumann M, Boo LM, Ackerman AH, Gallagher CJ. 2013. Immunogenicity of botulinum toxins. J Neural. Transm (Vienna). 120:275-290.
Nishiki T, Kamata Y, Nemoto Y, Omori A, Ito T, Takahashi M, Kozaki S. 1994. Identification of protein receptor for Clostridium botulinum type B neurotoxin in rat brain synaptosomes. J. 10 Biol. Chem. 269:10498-10503.
Nishiki T, Tokuyama Y, Kamata Y, Nemoto Y, Yoshida A, Sato K, Sekiguchi M, Takahashi M, Kozaki S. 1996. The high-affinity binding of Clostridium botulinum type B neurotoxin to synaptotagmin II associated with gangliosides GT1b/GD1a. FEBS Lett. 378:253-257.
Robert X, Gouet, P. 2014. Deciphering key features in protein structures with the new ENDscript server. Nucl. Acids Res. 42, W320-W324.
Rossetto O, Caccin P, Rigoni M, Tonello F, Bortoletto N, Stevens RC, Montecucco C. 2001. Active-site mutagenesis of tetanus neurotoxin implicates TYR-375 and GLU-271 in metalloproteolytic activity. Toxicon 39:1151-1159.
Rossetto O, Pirazzini M, Montecucco C. 2014. Botulinum neurotoxins: genetic, structural and mechanistic insights. Nat Rev Microbial. 2014; 12:535-549.
Rummel A, Mahrhold S, Bigalke H, Binz T. 2011. Exchange of the H(CC) domain mediating double receptor recognition improves the pharmacodynamic properties of botulinum neurotoxin. FEBS J. 278:4506-4515.
Rummel A. 2013. Double receptor anchorage of botulinum neurotoxins accounts for their exquisite neurospecificity. Curr Top Microbiol Immunol. 364:61-90.
Rummel A. 2016. Two Feet on the Membrane: Uptake of Clostridial Neurotoxins. Curr Top Microbiol Immunol. Springer, Berlin, Heidelberg.
Schengrund C. L., DasGupta B. R. and Ringler N. J. 1991. Binding of botulinum and tetanus neurotoxins to ganglioside GT1b and derivatives thereof. J. Neurochem. 57, 1024-1032.
Schiavo G, Matteoli M, Montecucco C. 2000. Neurotoxins affecting exocytosis. Physiol. Rev. 80:717-766.
Shone CC, Hambleton P, Melling J. 1985. Inactivation of Clostridium botulinum type A neurotoxin by trypsin and purification of two tryptic fragments. Proteolytic action near the COOH-terminus of the heavy subunit destroys toxin-binding activity. Eur J Biochem 151, 75-82.
Sievers F, Wilm A, Dineen DG, Gibson TJ, Karplus K, Li W, Lopez R, McWilliam H, Remmert M, Soding J, Thompson JD, Higgins DG. 2011. Fast, scalable generation of high-quality protein multiple sequence alignments using Clustal Omega. Molecular Systems Biology 7:539.
Stenmark P, Dupuy J, Imamura A, Kiso M, Stevens RC. 2008. Crystal structure of botulinum neurotoxin type A in complex with the cell surface co-receptor GTIb-insight into the toxin-neuron interaction. PLoS Pathog. 4:e1000129.
Strotmeier, J., Willjes, G., Binz, T. & Rummel, A. 2012. Human synaptotagmin-II is not a high affinity receptor for botulinum neurotoxin Band G: increased therapeutic dosage and immunogenicity. FEBS Lett. 586, 310-313.
Sudhof TC, Rothman JE. 2009. Membrane fusion: grappling with SNARE and SM proteins. Science 323, 474-477.
Sutton JM, Wayne J, Scott-Tucker A, O'Brien SM, Marks PM, Alexander FC, Shone CC & Chaddock JA. 2005. Preparation of specifically activatable endopeptidase derivatives of Clostridium botulinum toxins type A, B, and C and their applications. Protein Expr Purif 40, 31-41.
Swaminathan S. 2011. Molecular structures and functional relationships in clostridial neurotoxins. FEBS J. 278:4467-4485.
Takamizawa K., Iwamori M., Kozaki S., Sakaguchi G., Tanaka R., Takayama H. and Nagai Y. 1986. TLC immunostaining characterization of Clostridium botulinum type A neurotoxin binding to gangliosides and free fatty acids. FEBS Lett. 201:229-232.
Takamori S, Holt M, Stenius K, Lemke EA, Grønborg M, Riedel D, Urlaub H, Schenck S, Brugger B, Ringler P, Muller SA, Rammner B, Grater F, Hub JS, De Groot BL, Mieskes G, Moriyama Y, Klingauf J, Grubmuller H, Heuser J, Wieland F, Jahn R. 2006. Molecular anatomy of a trafficking organelle. Cell 127:831-846.
Tao L, Peng L, Berntsson RP, Liu SM, Park S, Yu F, Boone C, Palan S, Beard M, Cha brier PE, Stenmark P, Krupp J, Dong M. 2017. Engineered botulinum neurotoxin B with improved efficacy for targeting human receptors. Nat Commun. 8:53.
Wang J, Meng J, Lawrence GW, Zurawski TH, Sasse A, Bodeker MO, Gilmore MA, Fernandez-Salas E, Francis J, Steward LE, Aoki KR, Dolly JO. 2008. Novel chimeras of botulinum neurotoxins A and E unveil contributions from the binding, translocation, and protease domains to their functional characteristics. J Biol Chem. 283:16993-17002.
Wang J, Zurawski TH, Bodeker MO, Meng J, Boddul S, Aoki KR, Dolly JO. 2012. Longer-acting and highly potent chimaeric inhibitors of excessive exocytosis created with domains from botulinum neurotoxin A and B. Biochem J. 444:59-67.
Wilhelm BG, Mandad S, Truckenbrodt S, Krohnert K, Schafer C, Rammner B, Koo SJ, Clagen GA, Krauss M, Haucke V, Urlaub H, Rizzoli SO. 2014. Composition of isolated synaptic boutons reveals the amounts of vesicle trafficking proteins. Science 344:1023-1028.
Willjes G, Mahrhold S, Strotmeier J, Eichner T, Rummel A, Binz T. 2013. Botulinum neurotoxin G binds synaptotagmin-II in a mode similar to that of serotype B: tyrosine 1186 and lysine 1191 cause its lower affinity. Biochemistry. 2013 52:3930-3938.
Yao G, Zhang S, Mahrhold S, Lam KH, Stern D, Bagramyan K, Perry K, Kalkum M, Rummel A, Dong M, Jin R. 2016. N-linked glycosylation of SV2 is required for binding and uptake of botulinum neurotoxin A. Nat Struct Mai Biol. 23:656-662.
Zhang S, Masuyer G, Zhang J, Shen Y, Lundin D, Henriksson L, Miyashita SI, Martinez-Carranza M, Dong M, Stenmark P. 2017. Identification and characterization of a novel botulinum neurotoxin. Nat Commun. 8:14130.
Stern D, Weisemann J, Le Blanc A, van Berg L, Mahrhold S, Piesker J, Laue M, Luppa PB, Dorner MB, Dorner BG, Rummel A. 2018. A lipid-binding loop of botulinum neurotoxin serotypes B, DC and G is an essential feature to confer their exquisite potency. PLoS Pathog. 14(5):e1007048.

1. A botulinum neurotoxin (BoNT) Heavy Chain Binding domain (HC/TAB) having a N-terminal end (HCN) and a C-terminal end (HCC), wherein the HC/TAB comprises:
a) a synaptotagmin (Syt) receptor binding site, and
b) a synaptic associated vesicle 2 (SV2) receptor binding site, and
c) a ganglioside (Gang) receptor binding site,
and wherein said HC/TAB is adapted to synergistically bind to a synaptotagmin (Syt) receptor, a synaptic associated vesicle 2 (SV2) receptor and a ganglioside (Gang) receptor.

2. The HC/TAB according to claim 1, wherein the sequences forming the Gang receptor binding site originates from any Gang receptor binding BoNT serotype and their subtypes.

3. The HC/TAB according to claim 1, wherein the sequences forming the Syt receptor binding site originates from any Syt receptor binding BoNT serotype and their subtypes.

4. The HC/TAB according to claim 1, wherein the sequences forming the SV2-receptor binding site originates from any SV2 receptor binding BoNT serotype and their subtypes.

5. (canceled)

6. The HC/TAB according to claim 1, characterized in that the HCC domain is composed interchangeably of sequences from BoNT serotype A (BoNT/A) and BoNT serotype B (BoNT/B).

7. The HC/TAB according to claim 1, characterized in that said HCC end is composed according to a sequence A1B1A2B2A3, wherein A indicates a sequence from BoNT/A and B indicates a sequence from BoNT/B.

8.-14. (canceled)

15. The HC/TAB according to claim 1, having an amino acid sequence which is at least 60% identical to the sequence of SEQ ID NO: 1.

16. A polypeptide comprising a botulinum neurotoxin (BoNT) Heavy Chain Binding domain (HC/TAB) having a N-terminal end (HCN) and a C-terminal end (HCC), wherein the HC/TAB comprises:
a) a synaptotagmin (Syt) receptor binding site, and
b) a synaptic associated vesicle 2 (SV2) receptor binding site, and
c) a ganglioside (Gang) receptor binding site,
and wherein said HC/TAB is adapted to synergistically bind to a synaptotagmin (Syt) receptor, a synaptic associated vesicle 2 (SV2) receptor and a ganglioside (Gang) receptor, coupled to any one or more other protein, polypeptide, amino acid sequence or fluorescent probe, directly or via a linker.

17. The polypeptide according to claim 16, wherein said polypeptide is a BoNT polypeptide (BoNT/TAB), characterized in that said BoNT/TAB in addition to the HC/TAB comprises a Heavy Chain Translocation domain (HN), a Light chain (LC) and a protease site positioned between the LC and HN in the polypeptide sequence, wherein the HN and the LC, respectively and independently of each other, originate from any of the BoNT serotypes A, B, C, D, DC, E, En, F, G or X and their subtypes.

18. The polypeptide according to claim 16, further comprising any other protein, polypeptide, amino acid sequence or fluorescent probe, linked thereto, directly or via a linker.

19. (canceled)

20. The polypeptide according to claim 16, having an amino acid sequence which is at least 60% identical to the sequence of any of SEQ ID NOs 3, 5, 6, 8, 10 and 12.

21.-22. (canceled)

23. A therapeutic method or a cosmetic method comprising administering to a subject the HC/TAB of claim 1.

24. The therapeutic or cosmetic method of claim 23, wherein the HC/TAB is administered to the subject to dampen and/or inactivate a muscle.

25. (canceled)

26. The therapeutic or cosmetic method of claim 23, wherein the subject has a disorder selected from the group consisting of: spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhydrosis, excessive salivation, excessive gastrointestinal secretions, secretory disorders, pain from muscle spasms, headache pain, sports injuries, and depression.

27.-32. (canceled)

33. A therapeutic or a cosmetic method comprising administering to a patient a polypeptide according to claim 16.

34. The therapeutic or cosmetic method according to claim 33, wherein the polypeptide is administered to a subject to dampen and/or inactivate a muscle.

35. The therapeutic or cosmetic method of claim 33, wherein the subject has a disorder selected from the group consisting of: spasmodic dysphonia, spasmodic torticollis, laryngeal dystonia, oromandibular dysphonia, lingual dystonia, cervical dystonia, focal hand dystonia, blepharospasm, strabismus, hemifacial spasm, eyelid disorder, cerebral palsy, focal spasticity and other voice disorders, spasmodic colitis, neurogenic bladder, anismus, limb spasticity, tics, tremors, bruxism, anal fissure, achalasia, dysphagia and other muscle tone disorders and other disorders characterized by involuntary movements of muscle groups, lacrimation, hyperhydrosis, excessive salivation, excessive gastrointestinal secretions, secretory disorders, pain from muscle spasms, headache pain, sports injuries, and depression.

36. A method for transporting a protein of interest to a neural cell, the method comprising coupling the protein to the HN or the LC of the polypeptide of claim 17, and administering the coupled protein and polypeptide to a subject in need thereof.
